(19) United States
(12) Patent Application Publication
(10) Pub. No.: US 2009/0112626 A1
(43) Pub. Date: Apr. 30, 2009

(54) REMOTE WIRELESS MONITORING, PROCESSING, AND COMMUNICATION OF PATIENT DATA

(76) Inventors: Cary Talbot, Santa Clarita, CA (US); Arieh S. Halpern, Beverly Hills, CA (US)

Correspondence Address: INGRASSIA FISHER & LORENZ, P.C., 7010 E. COCHISE ROAD, SCOTTSDALE, AZ, 85253, US

(21) Appl. No.: 11/929,988

(22) Filed: Oct. 30, 2007

(51) Int. Cl. G06Q 50/00; A61B 5/00

(52) U.S. Cl. 705/3; 600/300

(57) ABSTRACT

A remote wireless monitoring system for patient data, and an ambulatory system for processing and transmitting physiological characteristic data are provided. An embodiment of a system for remote wireless monitoring of data for a patient includes an ambulatory sensor/transmitter subsystem that wirelessly transmits measured values of a physiological characteristic of the patient, a base station that wirelessly receives signals from the sensor/transmitter subsystem, and a remote monitor that wirelessly receives signals from the base station. The remote monitor is configured to generate audio and/or visual indicia (representing alarms, the measured values, device or system status information, etc.) in response to the base station signals. An embodiment of an ambulatory system includes a physiological characteristic sensor, a self-contained sensor processor module coupled to the ambulatory physiological characteristic sensor, and an ambulatory data receiver device coupled to the self-contained sensor processor module.

### TECHNICAL FIELD

Embodiments of the subject matter described herein relate generally to medical device systems that handle physiological patient data. More particularly, embodiments of the subject matter relate to the remote wireless monitoring and wireless communication of physiological patient data and/or data related to the operation or status of medical device system components that process physiological patient data.

### BACKGROUND

Portable medical devices having wireless data communication capabilities are becoming increasingly popular, especially for patients that have conditions that must be monitored on a continuous or frequent basis. For example, diabetics are usually required to modify and monitor their daily lifestyle to keep their body in balance, in particular, their blood glucose (“BG”) levels. Individuals with Type 1 diabetes and some individuals with Type 2 diabetes use insulin to control their BG levels. To do so, diabetics routinely keep strict schedules, including ingesting timely nutritious meals, partaking in exercise, monitoring glucose levels daily, and adjusting and administering insulin dosages accordingly.

The prior art includes a number of insulin pump systems that are designed to deliver accurate and measured doses of insulin via infusion sets (an infusion set delivers the insulin through a small diameter tube that terminates at a cannula inserted under the patient's skin). In lieu of a syringe, the patient can simply activate the insulin pump to administer an insulin bolus as needed, for example, in response to the patient's current glucose level. A patient can measure his glucose level using a glucose measurement device, such as a test strip meter, a continuous glucose measurement system, or the like. Glucose measurement devices use various methods to measure the glucose level of a patient, such as a sample of the patient's blood, a sensor in contact with a bodily fluid, an optical sensor, an enzymatic sensor, or a fluorescent sensor. When the measurement device has generated a glucose measurement, the value is displayed on the measurement device. A continuous glucose monitoring system can monitor the patient's glucose level in real time.

Insulin pumps and continuous glucose monitoring devices may also be configured to communicate with remote control devices, monitoring or display devices, BG meters, and other devices associated with such an infusion system. Individual devices within conventional infusion systems may be configured to support a limited amount of wired or wireless data communication to support the operation of the infusion system. For example, a continuous glucose monitoring sensor may include a wireless transmitter that communicates with a glucose monitor device or an insulin pump within the infusion system. Moreover, an insulin pump device itself may include a display and monitoring functions for pump-related and/or patient-related data and alarms.

### BRIEF SUMMARY

A system that performs remote wireless monitoring of a physiological characteristic of a patient (such as glucose level), an ambulatory telemetry subsystem suitable for use with such a system, and related operating methods are provided. The embodiments of the systems and methods provided herein facilitate the wireless transmission of patient data and/or operating status data of the system components within a local setting such as a dwelling or a building. Moreover, certain embodiments of the systems and methods provided herein facilitate transmission of patient data and/or operating status data of the system components via an external data communication network.

The above and other aspects may be carried out by an embodiment of a system for remote wireless monitoring of data for a patient. The system includes: an ambulatory sensor/transmitter subsystem configured to obtain measured values of a physiological characteristic of the patient, and to wirelessly transmit sensor signals that convey the measured values; a base station in wireless communication with the ambulatory sensor/transmitter subsystem, the base station being configured to wirelessly receive the sensor signals, generate base station signals in response to the sensor signals, and wirelessly transmit the base station signals; and a remote monitor in wireless communication with the base station. The remote monitor is configured to wirelessly receive the base station signals, and generate audio/visual indicia in response to the base station signals.

The above and other aspects may be carried out by another embodiment of a system for remote wireless monitoring of data for a patient. This system includes: an ambulatory telemetry device configured to wirelessly transmit physiological characteristic data for the patient; a base station in wireless communication with the ambulatory telemetry device, the base station being configured to wirelessly receive the physiological characteristic data, generate base station signals in response to the physiological characteristic data, and wirelessly transmit the base station signals; and a remote monitor in wireless communication with the base station. The remote monitor is configured to wirelessly receive the base station signals, and generate audio/visual indicia in response to the base station signals.

The above and other aspects may be carried out by another embodiment of a system for remote wireless monitoring of data for a patient. This system includes: a transmitting device configured to wirelessly transmit patient data signals that convey measured values of a physiological characteristic of the patient; and a plurality of wireless remote units for the transmitting device. The plurality of wireless remote units are cooperatively configured to operate as a wireless repeater network for the patient data signals. Moreover, the plurality of wireless remote units includes a wireless remote monitor in communication with the transmitting device, the wireless remote monitor being configured to wirelessly receive the patient data signals or retransmitted versions thereof, and generate audio/visual indicia of the measured values.

The above and other aspects may be carried out by an embodiment of a method for remote wireless monitoring of data for a patient. The method involves: measuring a physiological characteristic of the patient; wirelessly transmitting a sensor signal that conveys a measured value of the physiological characteristic; wirelessly receiving the sensor signal at a base station; generating a base station signal in response to the sensor signal; wirelessly transmitting the base station signal from the base station; and wirelessly receiving the base station signal, or a retransmitted version thereof, at a remote monitor.

The above and other aspects may be carried out by an embodiment of a wireless repeater for a system that remotely monitors patient data. The wireless repeater includes: a receiver configured to wirelessly receive sensor signals that convey measured values of a physiological characteristic of a patient; a transmitter coupled to the receiver, and configured to wirelessly retransmit the sensor signals; a signal analyzer coupled to the receiver, and configured to analyze characteristics and content of received signals; a wireless signal strength indicator coupled to the signal analyzer, and configured to generate audio/visual indicia of received signal strength while the wireless repeater is operating in a setup mode; and a data throughput indicator coupled to the signal analyzer, and configured to generate audio/visual indicia of wirelessly received data while the wireless repeater is operating in the setup mode.

The above and other aspects may be carried out by an embodiment of an ambulatory system for processing physiological characteristic data for a patient. The system includes: an ambulatory physiological characteristic sensor configured to generate electrical signals that are indicative of a physiological characteristic of the patient; a self-contained sensor processor module coupled to the ambulatory physiological characteristic sensor, the self-contained sensor processor module being configured to receive the electrical signals from the ambulatory physiological characteristic sensor, and generate measured values of the physiological characteristic from the electrical signals; and an ambulatory telemetry device coupled to the self-contained sensor processor module, the ambulatory telemetry device being configured to receive the measured values, generate sensor signals that convey the measured values, and wirelessly transmit the sensor signals for reception at a destination device.

The above and other aspects may be carried out by another embodiment of an ambulatory system for processing physiological characteristic data for a patient. This system includes: an ambulatory physiological characteristic sensor configured to generate electrical signals that are indicative of a physiological characteristic of the patient; a self-contained sensor processor module coupled to the ambulatory physiological characteristic sensor, the self-contained sensor processor module being configured to receive the electrical signals from the ambulatory physiological characteristic sensor, and generate measured values of the physiological characteristic from the electrical signals; and an ambulatory monitor device coupled to the self-contained sensor processor module, the ambulatory monitor device being configured to receive the measured values, and produce audio/visual indicia associated with the measured values.

The above and other aspects may be carried out by an embodiment of a system comprising an ambulatory physiological characteristic sensor, an ambulatory data receiver device, and a self-contained sensor processor module coupled between the ambulatory physiological characteristic sensor and the ambulatory data receiver device. The system performs an embodiment of a method for communicating physiological characteristic data for a patient. The method involves: generating, with the ambulatory physiological characteristic sensor, electrical signals that are indicative of a physiological characteristic of the patient; receiving the electrical signals at the self-contained sensor processor module; generating, with the self-contained sensor processor module, measured values of the physiological characteristic from the electrical signals; and receiving the measured values at the ambulatory data receiver.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a diagram that depicts an embodiment of a system for remote wireless monitoring of patient data as deployed within a dwelling;

FIG. 2 is a schematic representation of an embodiment of a sensor/transmitter unit suitable for use with a remote wireless monitoring system;

FIG. 3 is a schematic representation of a device that represents an embodiment of an ambulatory telemetry device, an embodiment of a base station, or an embodiment of a remote monitor suitable for use with a remote wireless monitoring system;

FIG. 4A is a schematic representation of an embodiment of a repeater suitable for use with a remote wireless monitoring system;

FIG. 4B is a perspective view of an embodiment of a repeater suitable for use with a remote wireless monitoring system;

FIG. 5 is a schematic representation of an alternate embodiment of a system for remote wireless monitoring of patient data;

FIG. 6 is a schematic representation of another alternate embodiment of a system for remote wireless monitoring of patient data;

FIG. 7 is a schematic representation of yet another alternate embodiment of a system for remote wireless monitoring of patient data;

FIG. 8 is a schematic representation of an embodiment of a system for communicating and processing physiological characteristic data for a patient;

FIG. 9 is a schematic representation of an embodiment of an ambulatory system for communicating and processing physiological characteristic data for a patient;

FIG. 10 is a schematic representation of an embodiment of a sensor processor module suitable for use with an ambulatory system for communicating and processing physiological characteristic data for a patient;

FIG. 11 is a schematic representation of an alternate embodiment of an ambulatory system for communicating and processing physiological characteristic data for a patient; and

FIG. 12 is a schematic representation of an embodiment of an ambulatory data receiver device suitable for use with an ambulatory system for communicating and processing physiological characteristic data for a patient.

### DETAILED DESCRIPTION

The following detailed description is merely illustrative in nature and is not intended to limit the embodiments of the invention or the application and uses of such embodiments. Furthermore, there is no intention to be bound by any expressed or implied theory presented in the preceding technical field, background, brief summary or the following detailed description.

Techniques and technologies may be described herein in terms of functional and/or logical block components, and with reference to symbolic representations of operations, processing tasks, and functions that may be performed by various computing components or devices. Such operations, tasks, and functions are sometimes referred to as being computer-executed, computerized, software-implemented, or computer-implemented. In practice, one or more processor devices can carry out the described operations, tasks, and functions by manipulating electrical signals representing data bits at memory locations in the system memory, as well as other processing of signals. The memory locations where data bits are maintained are physical locations that have particular electrical, magnetic, optical, or organic properties corresponding to the data bits. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or instructions that perform the various tasks. The program or code segments can be stored in a processor-readable medium or transmitted by a computer data signal embodied in a carrier wave over a transmission medium or communication path. The “processor-readable medium” or “machine-readable medium” may include any medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like.

The following description refers to elements or nodes or features being “connected” or “coupled” together. As used herein, unless expressly stated otherwise, “connected” means that one element/node/feature is directly joined to (or directly communicates with) another element/node/feature, and not necessarily mechanically. Likewise, unless expressly stated otherwise, “coupled” means that one element/node/feature is directly or indirectly joined to (or directly or indirectly communicates with) another element/node/feature, and not necessarily mechanically.

For the sake of brevity, conventional techniques related to infusion system operation, insulin pump and/or infusion set operation, blood glucose sensing and monitoring, signal processing, data transmission, signaling, network control, and other functional aspects of the systems (and the individual operating components of the systems) may not be described in detail here. Examples of infusion pumps and/or communication options may be of the type described in, but not limited to, U.S. Pat. Nos. 4,562,751; 4,685,903; 5,080,653; 5,505,709; 5,097,122; 6,554,798; 6,558,320; 6,558,351; 6,641,533; 6,659,980; 6,752,787; 6,817,990; and 6,932,584, which are herein incorporated by reference. Examples of glucose sensing and/or monitoring devices maybe be of the type described in, but not limited to, U.S. Pat. Nos. 6,484,045; 6,809,653; 6,892,085; and 6,895,263, which are herein incorporated by reference. Furthermore, the connecting lines shown in the various figures contained here are intended to represent example functional relationships and/or physical couplings between the various elements. It should be noted that many alternative or additional functional relationships or physical connections may be present in an embodiment of the described subject matter.

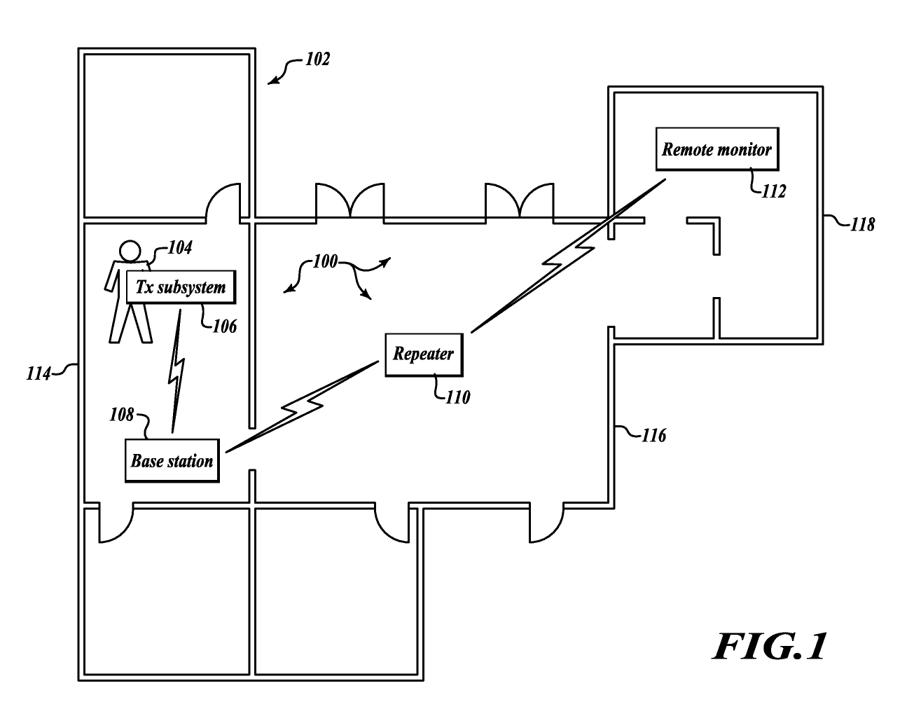

FIG. 1 is a diagram that depicts an embodiment of a system 100 for remote wireless monitoring of data within a dwelling 102. As used here, a “dwelling” is any physical structure that can be occupied by a patient. This includes but is not limited to free-standing structures, multiple unit structures (e.g., duplex, condominium, townhouse, apartments), hotels or motels, boats, airplanes, spaceships, space stations, remote interstellar plant habitats, etc. For this embodiment, system 100 is configured to monitor at least one physiological characteristic of a patient 104. System 100 may be alternatively or additionally configured to perform remote wireless monitoring of other data types, such as operating status data of one or more of components of system 100. As used here, a “physiological characteristic” is any detectable, observable, or measurable quantity, parameter, condition, status, or the like, that is associated with the biological functioning of patient 104. For example, glucose level, blood oxygen level, heart rate, and blood pressure represent different physiological characteristics that could be handled by system 100. The various systems described here, including system 100, are suitably configured to process and communicate glucose level data. However, embodiments of these systems can be alternately configured to support other monitored physiological characteristics.

System 100 generally includes, without limitation: an ambulatory sensor/transmitter subsystem 106; a base station 108; a repeater 110; and a remote monitor 112. FIG. 1 depicts patient 104, ambulatory sensor/transmitter subsystem 106, and base station 108 in a first room 114 of the dwelling 102, depicts repeater 110 in a second room 116 of the dwelling 102, and depicts remote monitor 112 in a third room 118 of the dwelling. FIG. 1 also depicts an operating state where ambulatory sensor/transmitter subsystem 106 is in wireless communication with base station 108, which is in wireless communication with repeater 110, which is in wireless communication with remote monitor 112. In practice, the components of system 100 can be configured to wirelessly communicate with each other in any fashion (subject to transmit/receive ranges, supported data communication protocols, transmit power levels, and the like). Thus, under certain conditions ambulatory sensor/transmitter subsystem 106 could wirelessly communicate directly with repeater 110 and/or directly with remote monitor 112. Likewise, under certain conditions base station 108 could wirelessly communicate directly with remote monitor 112.

System 100 depicted in FIG. 1 represents a relatively simple implementation that employs one base station 108, one repeater 110, and one remote monitor 112, all operating within the general confines of dwelling 102. Ambulatory sensor/transmitter subsystem 106 wirelessly transmits sensor signals in compliance with a relatively short range wireless data communication protocol, such as WMTS or BLUETOOTH®, and base station 108 wirelessly receives the sensor signals in compliance with the same relatively short range wireless data communication protocol. In contrast, base station 108 wirelessly transmits base station signals in compliance with a relatively long range wireless data communication protocol, such as IEEE 802.11, and repeater 110 wirelessly receives the base station signals in compliance with the same relatively long range wireless data communication protocol. Likewise, repeater 110 wirelessly retransmits the base station signals in compliance with the same relatively long range wireless data communication protocol, and remote monitor 112 wirelessly receives the retransmitted base station signals in compliance with the same relatively long range wireless data communication protocol.

System 100 (and other embodiments described here) facilitates the remote wireless monitoring of patient data and/or system data at remote monitor 112. System 100 provides the ability to remotely monitor the patient's glucose values continuously, and system 100 can be configured to automatically generate alarms for patient-triggered events (e.g., a hypoglycemic or a hyperglycemic event) and/or for operational device-triggered events. Notably, these alarms can be generated from: any location within the dwelling 102 using, for example, WiFi wireless data communication techniques; outside the dwelling 102 in an open area using cellular data communication techniques; or anywhere around the world using cellular and/or internet data communication techniques.

Ambulatory sensor/transmitter subsystem 106 is “ambulatory” in the sense that it is designed to be worn, carried, or attached to patient 104 in a manner that allows it to move about from place to place along with patient 104. Ambulatory sensor/transmitter subsystem 106 is suitably configured to obtain measured values of a physiological characteristic (e.g., glucose level) of patient 104, and to wirelessly transmit sensor signals that convey the measured values. Ambulatory sensor/transmitter subsystem 106 utilizes a relatively short range (low transmit power) wireless data communication protocol to transmit the sensor signals and/or other outgoing data. The short range wireless data communication protocol may be, without limitation: BLUETOOTH®; wireless medical telemetry (WMTS); amplitude modulated or frequency modulated radio communication using standard or proprietary protocols; or the like. The use of relatively low transmit power is preferred for use with a portable, battery powered, ambulatory sensor/transmitter subsystem 106.

As described in more detail below, ambulatory sensor/transmitter subsystem 106 includes one or more components that cooperate to support the particular system deployment. For example, a first embodiment of ambulatory sensor/transmitter subsystem 106 includes a physiological characteristic sensor and a sensor transmitter coupled to the physiological characteristic sensor (the sensor transmitter may be combined with the physiological characteristic sensor as an integrated unit). In such an embodiment, the sensor transmitter wirelessly transmits the sensor signals for reception at a destination device.

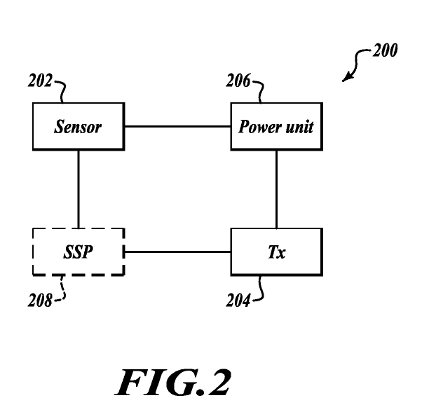

FIG. 2 is a schematic representation of an embodiment of a sensor/transmitter unit 200 suitable for use with the first embodiment of ambulatory sensor/transmitter subsystem 106. Sensor/transmitter unit 200 includes, without limitation, a physiological characteristic sensor 202, a sensor transmitter 204, and a power unit 206 coupled to physiological characteristic sensor 202 and to sensor transmitter 204. For this example, sensor/transmitter unit 200 also includes a sensor signal processor (SSP) 208—the dashed lines in FIG. 2 indicate the optional nature of SSP 208. Power unit 206, which may be realized as a replaceable or rechargeable battery, or as a non-replaceable battery in a disposable transmitter implementation, provides the operating power for physiological characteristic sensor 202, sensor transmitter 204, and SSP 208.

For this example, physiological characteristic sensor 202 is used for the continuous detection of patient glucose levels. Suitable sensor types include, without limitation: subcutaneous interstitial fluid contacting sensors; direct blood contacting sensors; non-invasive sensors; or ocular fluid contacting sensors. In practice, physiological characteristic sensor 202 is configured to generate electrical signals having voltages and/or currents that are indicative of the glucose level of the patient. For the first embodiment of ambulatory sensor/transmitter subsystem 106, SSP 208 processes these raw electrical signals into sensor signals that convey the measured glucose values. Thereafter, sensor transmitter 204 wirelessly transmits the sensor signals. In practice, SSP 208 converts the raw sensor data (i.e., electrical signals) as detected by physiological characteristic sensor 202, and derives glucose values for the patient from the raw electrical sensor signals. Other information may be transmitted from the ambulatory sensor/transmitter system, such as, without limitation: alarm commands; error codes; warnings; device ID; and/or patient ID. The ambulatory sensor/transmitter subsystem may contain algorithms to analyze the physiologic characteristic, such as, without limitation: high or low glucose levels; rising or falling glucose levels; predicted high or low glucose levels; and the like. These algorithms can generate alert or alarm messages or commands for transmission to other devices. Alternatively, the alarm algorithms may reside in another device such as the base station, remote monitor, or a repeater.

Sensor transmitter 204 is configured to wirelessly transmit the sensor signals to an appropriate destination device, such as base station 108. In certain embodiments, sensor transmitter 204 is a self contained transceiver that is attached to physiological characteristic sensor 202. Sensor transmitter 204 is capable of wirelessly transmitting: the raw unprocessed electrical sensor signals (if SSP 208 is not used); the processed sensor signals (if SSP 208 is embodied in sensor/transmitter unit 200); sensor operational status data; operational status data for SSP 208, if implemented; and operational power level of power unit 206. This information can be continuously transmitted to remote monitor 112 via base station 108 and/or repeater 110. Sensor transmitter 204 can be designed as a modular unit to facilitate the ability to interchange various types of wireless radio frequency (RF) units, thereby allowing sensor transmitter 204 to be configured for operation on different radio frequencies per the host country requirements and/or the use of various types of wireless communication technologies (e.g., BLUETOOTH®, IEEE 802.11, infrared, cellular, etc).

A second embodiment of ambulatory sensor/transmitter subsystem 106 includes a physiological characteristic sensor, a sensor transmitter coupled to the physiological characteristic sensor, and a telemetry device in wireless communication with the sensor transmitter. In this embodiment, the sensor transmitter is configured to wirelessly transmit sensor origination signals, and the telemetry device is configured to wirelessly receive the sensor origination signals, generate the sensor signals from the sensor origination signals, and wirelessly transmit the sensor signals for reception at a destination device. This second embodiment of ambulatory sensor/transmitter subsystem 106 is desirable when the wireless transmit range of the sensor transmitter is limited—the telemetry device has a longer wireless transmit range than the sensor transmitter. The second embodiment of ambulatory sensor/transmitter subsystem 106 may utilize a sensor/transmitter unit (such as sensor/transmitter unit 200) in conjunction with a suitably configured telemetry device. In such an embodiment, an SSP may be implemented in the sensor/transmitter unit, the telemetry device, or in a distributed manner in both. Alternatively, the SSP may be implemented elsewhere in system 100. The raw electrical sensor signals can be transmitted by sensor transmitter 204 to the telemetry device, or the raw electrical sensor signals can be initially processed by SSP 208 prior to transmission by sensor transmitter 204.

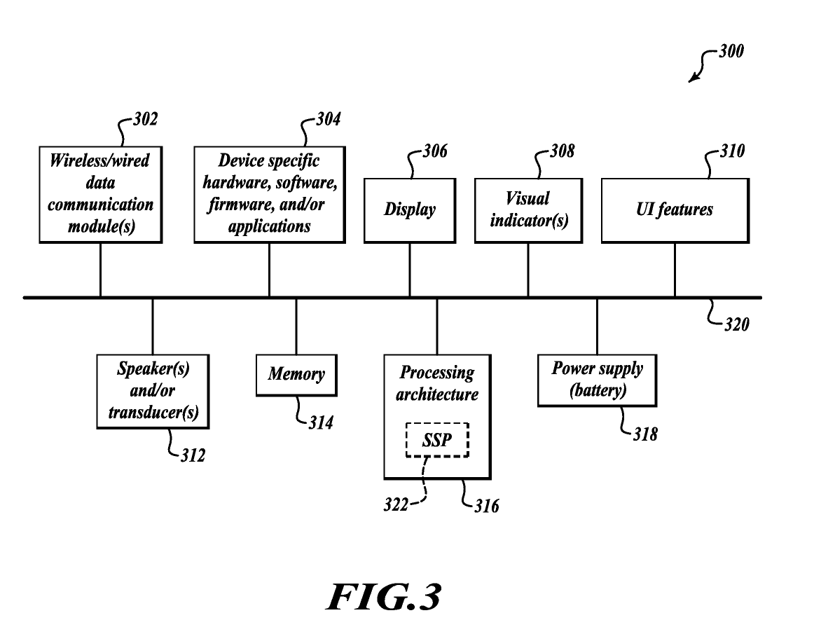

FIG. 3 is a schematic representation of a device 300 that represents an embodiment of an ambulatory telemetry device suitable for use with system 100. As mentioned above, the second embodiment of ambulatory sensor/transmitter subsystem 106 includes an ambulatory telemetry device together with sensor/transmitter unit 200. For the illustrated embodiment, device 300 includes: wireless/wired data communication module(s) 302; device specific hardware, software, firmware, and/or applications 304; a display element 306; one or more visual indicators 308; one or more user interface (UI) features 310; one or more speakers and/or transducers 312; a suitable amount of memory 314; a processing architecture 316; and a rechargeable or replaceable power supply, such as a battery 318. The elements of device 300 may be coupled together via a bus 320 or any suitable interconnection architecture.

Processing architecture 316 may be implemented or performed with a general purpose processor, a content addressable memory, a digital signal processor, an application specific integrated circuit, a field programmable gate array, any suitable programmable logic device, discrete gate or transistor logic, discrete hardware components, or any combination designed to perform the functions described here. A processor may be realized as a microprocessor, a controller, a microcontroller, or a state machine. Moreover, a processor may be implemented as a combination of computing devices, e.g., a combination of a digital signal processor and a microprocessor, a plurality of microprocessors, one or more microprocessors in conjunction with a digital signal processor core, or any other such configuration. Notably, ambulatory telemetry device 300 may include an optional SSP 322 (the dashed lines in FIG. 3 indicate its optional nature), which may be implemented in processing architecture 316. SSP 322 has the characteristics and functionality of SSP 208 (see FIG. 2).

Memory 314 may be realized as RAM memory, flash memory, EPROM memory, EEPROM memory, registers, a hard disk, a removable disk, a CD-ROM, or any other form of storage medium known in the art. In this regard, memory 314 can be coupled to processing architecture 316 such that processing architecture 316 can read information from, and write information to, memory 314. In the alternative, memory 314 may be integral to processing architecture 316. As an example, processing architecture 316 and memory 314 may reside in an ASIC.

Device-specific hardware, software, firmware, and/or applications 304 may vary from one embodiment of device 300 to another. For example, an ambulatory telemetry device can be implemented in different formats to address the needs of the particular application. In this regard, an ambulatory telemetry device may be configured as a telemetry-only unit, a telemetry unit combined and integrated with an ambulatory monitor device, a telemetry unit combined and integrated with an ambulatory fluid infusion device (e.g., an insulin pump), a telemetry unit combined and integrated with an ambulatory monitor device and an ambulatory fluid infusion device, etc. Thus, a combined ambulatory telemetry/monitor device is configured to perform wireless data telemetry, and produce audio/visual indicia associated with the measured values of the physiological characteristic, where such indicia may represent, without limitation: a text or graphical display of the measured values; alarms or alerts triggered by the measured values; patient instructions; or operating status data for one or more components of the remote wireless monitoring system. On the other hand, a combined ambulatory telemetry/pump device is configured to perform wireless data telemetry, and deliver fluid (such as insulin) to the patient as needed. Accordingly, device-specific hardware, software, firmware, and/or applications 304 will support telemetry functions and features, monitor functions and features (when device 300 includes monitor device functionality), and fluid infusion pump functions and features (when device 300 includes fluid infusion device functionality). Of course, device-specific hardware, software, firmware, and/or applications 304 may additionally or alternatively support other features and functionality, such as, without limitation: physiological characteristic meter functionality; alarm clock functionality; or the like. In practice, certain portions or aspects of device-specific hardware, software, firmware, and/or applications 304 may be implemented in one or more of the other blocks depicted in FIG. 3.

An embodiment of device 300 may employ any number of wireless and/or wired data communication modules 302. These data communication modules are suitably configured to support wireless/wired data communication (unidirectional or bidirectional, depending upon the particular implementation) between device 300 and other devices in the remote wireless monitoring system, for example, sensor transmitter 204 (see FIG. 2), base station 108, repeater 110, or remote monitor 112 (see FIG. 1).

A wireless data communication module is configured to support one or more wireless data communication protocols. Any number of suitable wireless data communication protocols, techniques, or methodologies may be supported by ambulatory telemetry device 300, including, without limitation: RF; IrDA (infrared); BLUETOOTH®; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; cellular/wireless/cordless telecommunication protocols; wireless home network communication protocols; paging network protocols; magnetic induction; satellite data communication protocols; wireless hospital or health care facility network protocols such as those operating in the WMTS bands; GPRS; and proprietary wireless data communication protocols such as variants of Wireless USB. In an embodiment of device 300, a wireless data communication module may include or be realized as hardware, software, and/or firmware, such as an RF front end, a suitably configured radio module (which may be a stand alone module or integrated with other or all functions of the device), a wireless transmitter, a wireless receiver, a wireless transceiver, an infrared sensor, an infrared diode and sensor, an electromagnetic transducer, or the like. Moreover, device 300 may include one or more antenna arrangements that cooperate with the wireless data communication module.

A wired data communication module supports data transfer over a cable, a wired connection, or other physical link. A wired data communication module is configured to support one or more wired/cabled data communication protocols. Any number of suitable data communication protocols, techniques, or methodologies may be supported by device 300, including, without limitation: Ethernet; home network communication protocols; USB; IEEE 1394 (Firewire); hospital network communication protocols; and proprietary data communication protocols. In an embodiment of device 300, a wired data communication module may include or be realized as hardware, software, and/or firmware, such as a suitably configured and formatted port, connector, jack, plug, receptacle, socket, adaptor, or the like.

Device 300 may also be designed to accommodate UI features 310 that allow the user to control the operation of device 300 and/or other devices within the system. UI features 310 may include a keypad, keys, buttons, switches, knobs, a touchpad, a joystick, a pointing device, a virtual writing tablet, or any device, component, or function that enables the user to select options, input information, or otherwise control the operation of device 300 and/or other devices within the system.

Display element 306 is suitably configured to enable device 300 to render and display information such as measured values of the physiological characteristic, alarms, device status information, clock/calendar data, and/or other information and data received or processed by device 300. Display element 306 is optional because certain embodiments of an ambulatory telemetry device need not display any information or data to the patient. Notably, the specific configuration, operating characteristics, size, resolution, and functionality of display element 306 can vary depending upon the practical implementation of device 300. For example, display element 306 may be implemented using a liquid crystal display (LCD), a plasma monitor, a stylus writing screen, a touchpad, or the like.

In addition to (or in lieu of) display element 306, device 300 may include one or more visual indicators 308, e.g., lights, mechanically actuated buttons, or the like. Visual indicators 308 can be utilized to generate a visually perceptible representation of an alarm, a reminder, an operating status of device 300 (or another device within the wireless remote monitoring system), a measured value of a physiological characteristic, or other visible indicia associated with the operation of device 300 and/or the operation of other devices within the remote wireless monitoring system.

Device 300 may also include one or more speakers or transducers 312, which serve as audio indicator(s). Speakers 312 can be utilized to generate audible alarms, reminder tones, audio messages, media clips, or other audible indicia associated with the operation of device 300 and/or the operation of other devices within the remote wireless monitoring system. For example, speakers 312 can be utilized to generate an audible representation of an operating status of device 300 (or another device within the wireless remote monitoring system), a measured value of a physiological characteristic, or the like.

Referring again to FIG. 1, base station 108 is a portable, tabletop, or wall mounted device that is preferably located in a room where patient 104 spends a significant amount of time (e.g., a bedroom or an office). The general configuration and functionality of device 300 (see FIG. 3) may also be utilized to implement base station 108. For use as a base station, however, certain components of device 300 (e.g., wireless/wired data communication modules 302, and device specific hardware, software, firmware, and/or applications 304) may need to be specifically configured to support the desired operation of base station 108.

Base station 108 represents a host device for system 100, and more than one base station 108 could be supported by system 100. Base station 108 is capable of being powered from an external power supply, such as a standard AC wall outlet, or from an internal battery. In certain embodiments, base station 108 uses discrete LED indicators to indicate the operational status and condition of base station 108, an alphanumeric display element to display the patient's glucose values, patient alarms, system alarms, etc., an audio speaker configured to emit audible alarm tones and other sounds, and UI features for operation of base station 108.

Base station 108 is suitably configured to wirelessly receive sensor signals transmitted by ambulatory sensor/transmitter subsystem 106, generate base station signals in response to the sensor signals, and wirelessly transmit the base station signals. A base station signal may convey or represent the measured values, alarm notifications, reminder notifications, device status data, or the like. An embodiment of base station 108 that utilizes an SSP will process raw electrical sensor signals (received from ambulatory sensor/transmitter subsystem 106) to derive the usable glucose values of the patient. The memory of base station 108 can be used for the storage and trending of the patient's glucose values, glucose alarm events, and system alarm events.

Certain embodiments of base station 108 include wireless and/or wired communication modules that are configured to facilitate data communication using an external communication network, e.g., a cellular telecommunication network, a wide area network, the Internet, a local area network, or the like. In contrast to ambulatory sensor/transmitter subsystem 106, base station 108 is suitably configured to wirelessly transmit its base station signals using a relatively long range wireless data communication protocol. The long range wireless data communication protocol may be, without limitation: any variant of IEEE 802.11; wireless USB; or the like. This extended range increases the likelihood that base station 108 will be able to wirelessly communicate with repeater 110 and/or remote monitor 112.

In practical embodiments, the data communication modules of base station 108 may include multipurpose PCMCIA ports that accommodate the insertion of various types of wireless communication cards for the wireless communication of the patient's glucose values and system operational status conditions to other devices. These other devices include, without limitation: repeater 110; remote monitor 112; or broadband wireless communication devices (e.g., a cellular telecommunication device, a personal digital assistant, or a mobile computing device).

For the embodiment depicted in FIG. 1, repeater 110 is in wireless communication with base station 108 (to wirelessly receive data from base station 108) and with remote monitor 112 (to wirelessly transmit data to remote monitor 112). Alternatively or additionally, repeater 110 could communicate directly with ambulatory sensor/transmitter subsystem 106. Repeater 110 is suitably configured to wirelessly receive base station signals generated by base station 108 and retransmit the base station signals to remote monitor 112, which receives the retransmitted versions of the base station signals. In this manner, repeater 110 functions to extend the transmission range of base station 108. In certain embodiments, repeater 110 is implemented as a small and self-contained unit that can be mounted on a wall, plugged into a standard AC wall socket, or placed on a table, shelf, mantle, bookcase, or the like.

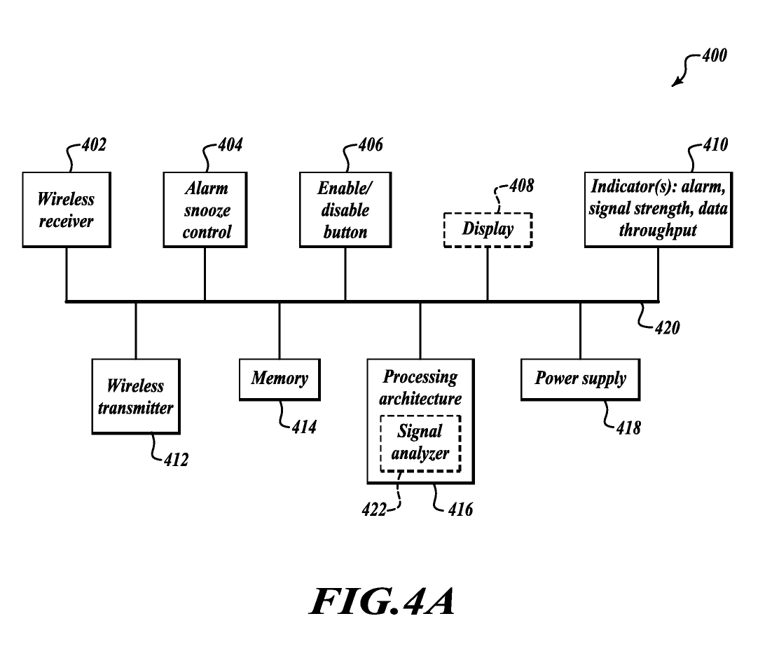

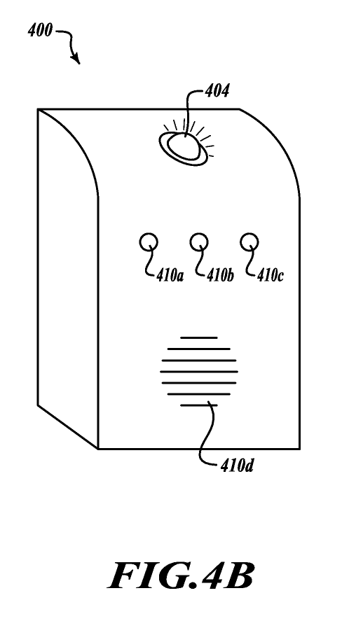

FIG. 4A is a schematic representation of an embodiment of a repeater 400 suitable for use with a remote wireless monitoring system such as system 100, and FIG. 4B is a perspective view of an embodiment of repeater 400. For the illustrated embodiment, repeater 400 includes: a wireless receiver 402; an alarm snooze control 404; an enable/disable button 406; a display element 408; one or more audio/visual indicators 410; a wireless transmitter 412; a suitable amount of memory 414; a processing architecture 416; and a power supply 418. The elements of repeater 400 may be coupled together via a bus 420 or any suitable interconnection architecture. Power supply 418 can be a rechargeable or replaceable backup power supply, such as a battery. Alternatively or additionally, power supply 418 can be configured to obtain operating power from a standard AC outlet.

Processing architecture 416 and memory 414 may be implemented or performed in the manner described above for processing architecture 316 and memory 314, respectively. Notably, repeater 400 may include a suitably configured signal analyzer 422, which may be implemented in processing architecture 416. The operation of signal analyzer 422 will be described in more detail below.

Repeater 400 utilizes wireless receiver 402 to wirelessly receive signals from base station 108 (or from other devices in system 100), and utilizes wireless transmitter 412 to wirelessly retransmit the received signals to one or more destination devices in system 100. In practice, wireless receiver 402 and wireless transmitter 412 may be implemented as one or more wireless data communication modules as generally described above in the context of wireless/wired data communication modules 302. Although not depicted in FIG. 4A, repeater 400 may also include one or more wired data communication modules.

Display element 408 is an optional element of repeater 400 (the dashed lines in FIG. 4A indicates the optional nature of display element 408). Display element 408 is optional because certain embodiments of repeater 400 need not display any information or data to the user. Display element 408, which can be generally configured as described above for display element 306, is suitably configured to enable repeater 400 to render and display information such as measured values of the physiological characteristic, alarms, device status information, clock/calendar data, and/or other information and data received or processed by repeater 400.

Signal analyzer 422 is suitably configured to analyze characteristics and/or content of received signals obtained by repeater 400. For this particular embodiment, signal analyzer 422 obtains or determines the received signal strength of received signals, and obtains or determines a data throughput quantifier associated with received signals. Signal analyzer 422 may indicate the received signal strength using any suitable technique or algorithm, and the manner in which the received signal strength is indicated may be simple or complex in nature. For instance, a simple methodology might compare the received signal strength to a threshold value, while a complex methodology might generate the actual signal strength values. Regarding data throughput, signal analyzer 422 may simply indicate whether or not any data of interest is being conveyed in the received signals. Alternatively, signal analyzer 422 may generate a data throughput value or quantity that indicates the amount of data currently passing through repeater 400.

Audio/visual indicators 410 may be generally configured as described above in the context of visual indicators 308 and speakers 312. Certain embodiments of repeater 400 use audio/visual indicators 410 as a wireless signal strength indicator, which is configured to generate audio and/or visual indicia of received signal strength while repeater 400 is operating. Thus, audio/visual indicators 410 are coupled to signal analyzer 422 such that they can be activated in response to the received signal strength determined by signal analyzer 422. Signal analyzer 422 and the signal strength indicator can be activated while repeater is operating in a setup mode to facilitate proper placement of repeater 400 within the intended system environment. While in the setup mode, a transmitting device generates a test signal for repeater 400, and the signal strength indicator will indicate (using a tone, a visual indicator, etc.) whether repeater 400 is receiving the test signal. Using the signal strength indicator the user can position repeater 400 in an appropriate location that maintains wireless coverage.

Certain embodiments of repeater 400 use audio/visual indicators 410 as a data throughput indicator, which is configured to generate audio and/or visual indicia of wirelessly received data while repeater 400 is operating. Thus, audio/visual indicators 410 are coupled to signal analyzer 422 such that they can be activated in response to the data throughput information determined by signal analyzer 422. Signal analyzer 422 and the data throughput indicator can be activated while repeater is operating in a setup mode to test whether repeater 400 is actually receiving data as intended. While in the setup mode, a transmitting device generates a test signal that conveys test data for repeater 400, and the data throughput indicator will indicate (using a tone, a visual indicator, etc.) whether repeater 400 is actually receiving the test data.

Certain embodiments of repeater 400 use audio/visual indicators 410 as an alarm indicator, which is configured to generate audio and/or visual alarms in response to received signals. In this regard, audio/visual indicators 410 are coupled to signal analyzer 422 such that they can be activated in response to received sensor signals that convey measured glucose values of the patient. These alarms can be triggered by the measured values, operating status information for repeater 400 or other devices in the system, low battery status, or any alarm-generating event described here in the context of a monitor device. Notably, repeater 400 need not provide any additional information related to the type of alarm, the alarm-triggering values, or other alarm-related details. Such additional information can be displayed at the source device and/or at a remote monitor in the system.

Embodiments of repeater 400 that include an alarm indicator may also employ alarm snooze control 404, which is configured to disable audio and/or visual alarms generated by repeater 400. Alarm snooze control 404 can be implemented as a hardware feature, a soft button (i.e., a graphical user interface feature), or any UI element that is designed to respond to a user-initiated command. When a user activates alarm snooze control 404, the alarm is silenced at repeater 400. Although not a requirement, repeater 400 may be configured to transmit, in response to the user activation of alarm snooze control 404, an alarm snooze signal to other devices in the system. Furthermore, the repeater may employ an alarm cancel control as described in more detail herein.

Referring to FIG. 4B, alarm snooze control 404 may be an illuminated snooze button that illuminates when an alarm is active. The embodiment shown in FIG. 4B includes an indicator 410a that when lit indicates that repeater 400 is in communication with the network, an indicator 410b that when lit indicates that repeater 400 is powered on, and an indicator 410c that when lit indicates that the user device is in communication with the network. This embodiment also includes an audio indicator 410d (e.g., a speaker or a transducer) that can be used to generate audible alarms, reminders, and/or notifications.

Repeater 400 and/or other components in the wireless remote monitoring system can generate an out-of-range alarm, which indicates that the ambulatory sensor/transmitter subsystem has lost wireless connectivity. Enable/disable button 406 is a UI feature of repeater 400 that, when activated by the user, temporarily disables alarms generated by the system for that particular user. This feature allows the patient to leave the premises without triggering an out-of-range alarm. Some embodiments may also utilize enable/disable button 406 to activate alarms that have been temporarily disabled by the user. Alternatively, alarms may be reactivated automatically upon detection of the ambulatory sensor/transmitter subsystem. In certain embodiments, automatic reactivation is enabled after a designated time delay. It should be appreciated that an equivalent enable/disable feature may also be supported by the ambulatory sensor/transmitter subsystem, base stations, and remote monitors.

Referring again to FIG. 1, remote monitor 112 is preferably implemented as a small transportable device which can take several forms—from a custom designed device for use within an enclosed dwelling, to an off the shelf smart phone that contains appropriate software applications that support operation in system 100. In this regard, remote monitor 112 can be in the form of a mobile computing device (e.g., a laptop computer), a mobile communication device (e.g., a cellular telephone, a personal digital assistant, or a portable video game device), or a bedside device that is small enough to fit on a nightstand.

Remote monitor 112 can be suitably configured to support a number of monitor-related operations, functions, and features. In this regard, remote monitor 112 (and other devices and components in system 100) may be implemented and configured as needed for the given deployment. For example, system 100 and the devices therein may employ the techniques, architectures, and technologies described in: U.S. patent application Ser. No. 11/413,268, publication number ______ (docket number 009.5003); U.S. patent application Ser. No. 11/583,344, publication number ______ (docket number 009.5005×1); U.S. patent application Ser. No. 11/671,174, publication number ______ (docket number 009.5005×2); and U.S. patent application Ser. No. 11/757,153, publication number ______ (docket number 009.5008); which are all incorporated herein by reference.

Remote monitor 112 is configured to wirelessly receive base station signals, repeater signals, and/or sensor SSP signals, (or retransmitted versions thereof) and, in response to the received base station signals, generate appropriate audio/visual indicia. This indicia may include an audio and/or visual representation of the measured values of the monitored physiological characteristic, an audio and/or visual representation of an alarm triggered by the measured values, an audio and/or visual representation of an operating status of ambulatory sensor/transmitter subsystem 106, an audio and/or visual representation of an operating status of base station 108, or the like. An embodiment of remote monitor 112 that utilizes an SSP will process raw electrical sensor signals (received from ambulatory sensor/transmitter subsystem 106, base station 108, or repeater 110) to derive the usable glucose values of the patient. The memory of remote monitor 112 can be used for the storage and trending of the patient's glucose values, glucose alarm events, and system alarm events.

More specifically, remote monitor 112 provides the ability to: remotely monitor the patient's glucose data continuously; display the glucose data either continuously, upon demand, or automatically upon detection of alarm event; enable the user to set upper and lower glycemic alarm limits; and upon the detection of an alarm event, trigger audible and/or visual alarms at remote monitor 112, thereby alerting the user to a patient and/or device alarm event. Remote monitor 112 also allows the user to view retrospective trend data in various formats, and performs other functions depending upon the application program. Upon the detection of a patient alarm (e.g., a hypoglycemic or hyperglycemic alarm event) and/or operational device alarm condition, remote monitor 112 triggers an alarm, and provides a displayed readout of the patient glucose value and upper and lower alarm limit values.

The general configuration and functionality of device 300 (see FIG. 3) may also be utilized to implement remote monitor 112. For use as a remote monitor, however, certain components of device 300 (e.g., UI features 310, display element 306, and device specific hardware, software, firmware, and/or applications 304) may need to be specifically configured to support the desired operation of remote monitor 112.

A network architecture such as that utilized by system 100 allows certain processing tasks and intelligence to be located in different physical components as needed or as desired. As one non-limiting example, the processing logic utilized to perform calibration of glucose measurements and the processing logic utilized to generate glucose level alarms may reside at one or more of the following components: ambulatory sensor/transmitter subsystem 106; base station 108; repeater 110; remote monitor 112. This type of flexibility allows a user or a network administrator to configure system 100 in an manner that best suits the needs of the particular deployment.

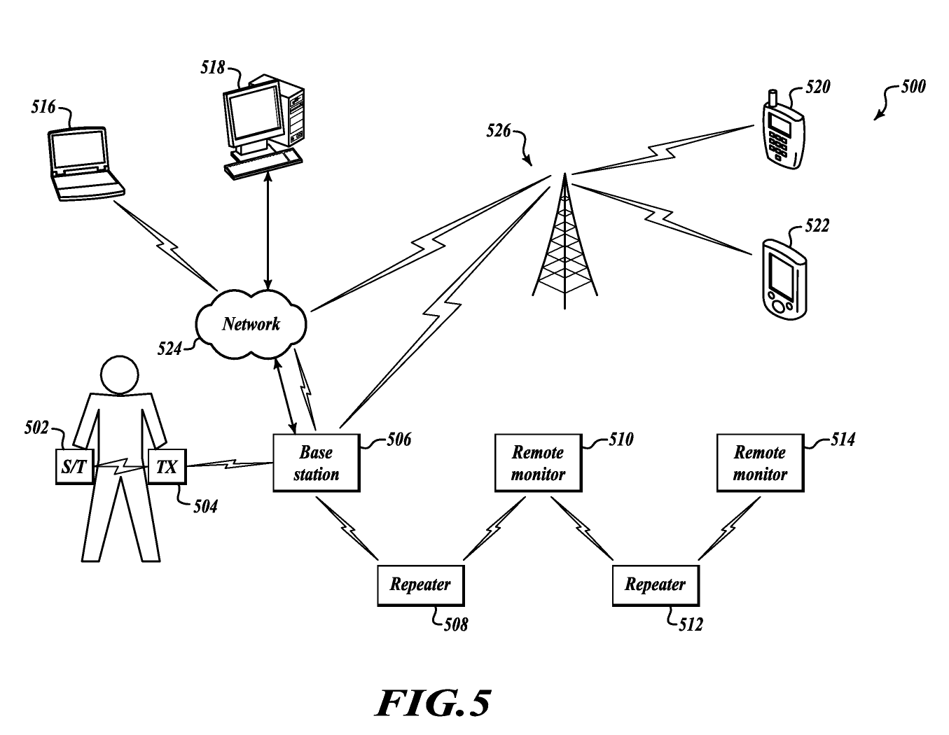

System 100 depicted in FIG. 1 represents only one possible implementation of a wireless remote monitoring system for patient data. FIGS. 5-7 depict alternate embodiments of such a system. The configuration and operation of some components in these alternate systems are similar to that described above in the context of system 100 and, therefore, the individual components will not be redundantly described with reference to FIGS. 5-7.

System 100 is a “local” system in that all of its components reside within dwelling 102. Moreover, the components of system 100 do not communicate with any external devices or external data communication networks. In contrast, FIG. 5 depicts a system 500 that contemplates data communication with one or more external devices. System 500 includes local devices, which may reside in a dwelling/building, and external devices that need not reside in the dwelling/building. The local devices include, without limitation: an ambulatory sensor/transmitter subsystem 502; an ambulatory telemetry device 504; a base station 506; a repeater 508; a remote monitor 510; a repeater 512; and a remote monitor 514. In this embodiment, the external devices include, without limitation: a laptop computer 516; a desktop computer 518; a remote monitor 520 having the form of a mobile communication device (e.g., a cellular telephone); and a remote monitor 522 having the form of a mobile computing device (e.g., a personal digital assistant, a smart phone, or a palmtop computer). Another suitable external device might be a remote monitor that displays data on a television screen. Such an external device could be used to send data on a phone line for display (similar to a caller ID display) on home phones or televisions.

System 500 employs a single base station 506, two repeaters 508/512, and two remote monitors 510/514 dispersed throughout the premises. In this embodiment, remote monitor 510 also includes the functionality of a wireless repeater—remote monitor 510 can retransmit its received data to one or more destination devices, such as repeater 512. Remote monitor 514 may be similarly configured to operate as a wireless repeater. As used here, base station 506, repeaters 508/512, and remote monitors 510/514 are considered to be wireless remote units for a transmitting device, such as ambulatory telemetry device 504. In this context, these wireless remote units are cooperatively configured to operate as a wireless repeater network for patient data signals and other data signals transmitted by the transmitting device. At least one of these wireless remote units takes the form of a wireless remote monitor (e.g., remote monitor 510 or remote monitor 514) that wirelessly receives patient data signals or retransmitted versions thereof, and generates audio/visual indicia in response to the received signals.

Base station 506 is capable of transmitting external network data communication signals to external devices. Such external network data communication signals can convey raw sensor data, measured values of the monitored physiological characteristic, status and/or operating data associated with components of system 500, or the like. In practice, an external network data communication signal can convey data via a LAN, a WAN, the Internet, a cellular telecommunication network, a satellite network, etc. For example, FIG. 5 depicts base station 506 communicating with laptop computer 516 and desktop computer 518 via a network 524. In this regard, base station 506 may communicate with network 524 using a dial-up connection, a DSL connection, a fiber optic link, a cable modem connection, or the like. Alternatively or additionally, base station 506 may communicate with network 524 using a wireless data communication protocol, as described above with reference to FIG. 3. In certain embodiments, base station 506 includes a cellular radio that supports wide area communication of data to remote monitors 520/522 via a cellular telecommunication network 526. This feature enables base station 506 to communicate with remote monitors 520/522 using cellular telecommunication data transmission techniques, technologies, and protocols. As depicted in FIG. 5, it may also be possible for a component in network 524 to communicate with remote monitors 520/522 via cellular telecommunication network 526.

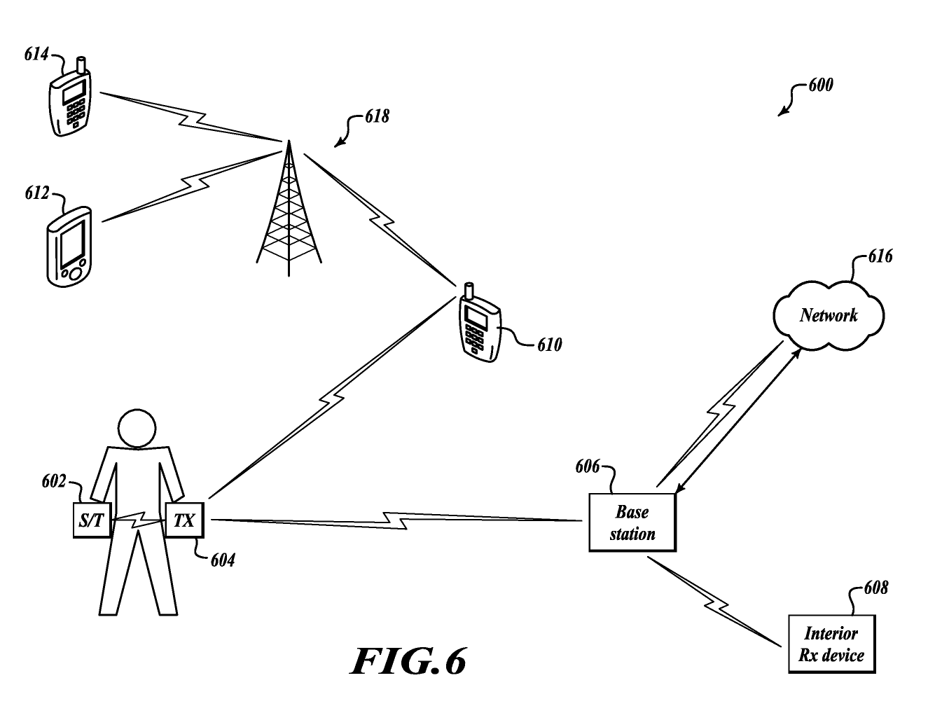

FIG. 6 is a schematic representation of another alternate embodiment of a system 600 for remote wireless monitoring of patient data. System 600 includes local devices, which may reside in a dwelling/building, and external devices that need not reside in the dwelling/building. The local devices include, without limitation: an ambulatory sensor/transmitter subsystem 602; an ambulatory telemetry device 604; a base station 606; and at least one interior receiving device 608 (e.g., a wireless repeater, a remote monitor, another base station, or any compatible device). In this embodiment, the external devices include, without limitation: a first remote monitor 610 having the form of a mobile communication device (e.g., a cellular telephone); a second remote monitor 612 having the form of a mobile computing device (e.g., a personal digital assistant, a smart phone, or a palmtop computer); and a third remote monitor 614 having the form of another mobile communication device.

Base station 606 is similar to base station 506 (FIG. 5) in that it supports data communication with an external network 616, which in turn can communicate with one or more destination devices (not shown in FIG. 6). Base station 606, however, need not be configured to communicate directly with a cellular data communication network 618. Rather, as depicted in FIG. 6, ambulatory telemetry device 604 is suitably configured to wirelessly communicate in a direct manner with remote monitor 610, which is realized as a cellular telephone. Notably, ambulatory telemetry device 604 wirelessly communicates with remote monitor 610 using a relatively short range wireless data communication protocol, such as BLUETOOTH® or wireless USB. In this embodiment, remote monitor 610 functions as a relay device by communicating with cellular data communication network 618. Thus, data transmitted by ambulatory telemetry device 604 can be received by remote monitor 610, reformatted if necessary, then retransmitted to remote monitor 612 and/or remote monitor 614 using cellular data communication network 618. It should be appreciated that the base station and the interior receiving device(s) in system 600 are optional. In other words, an alternate embodiment of system 600 can rely on the direct communication between ambulatory telemetry device 604 and remote monitor 610 in the absence of any in-dwelling components.

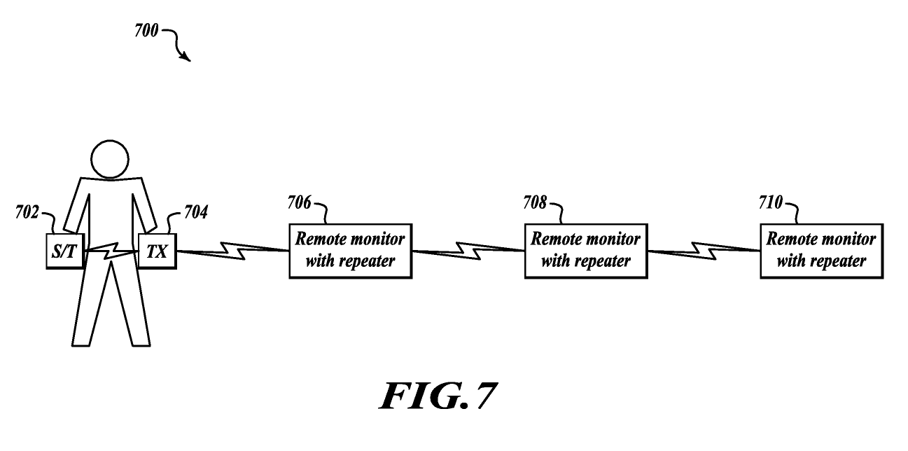

FIG. 7 is a schematic representation of yet another alternate embodiment of a system 700 for remote wireless monitoring of patient data. System 700 includes, without limitation: an ambulatory sensor/transmitter subsystem 702; an ambulatory telemetry device 704; and a plurality of wireless remote monitors, where each remote monitor also functions as a wireless repeater for ambulatory telemetry device 704. The illustrated embodiment includes remote monitors 706/708/710, although an embodiment of system 700 can include any number of such remote monitors. Remote monitors 706/708/710 are cooperatively configured to operate as a wireless repeater network for signals transmitted by ambulatory telemetry device 704. FIG. 7 depicts a simple scenario where ambulatory telemetry device 704 wirelessly communicates with remote monitor 706, which in turn wirelessly communicates with remote monitor 708, which in turn wirelessly communicates with remote monitor 710. In practice, however, each of these components may be capable of wirelessly communicating with any number of the other components. Moreover, at least one of the remote monitors 706/708/710 is utilized to generate audio/visual indicia in response to the received data. In other words, at least one of the remote monitors 706/708/710 functions as a monitor device for the physiological characteristic data, patient data, alarms, status information, reminders, and other data types described in more detail above.

An alternate embodiment of system 700 might employ one or more repeaters that do not have monitor functionality. Indeed, one possible system deployment includes ambulatory sensor/transmitter subsystem 702, ambulatory telemetry device 704, and one or more repeaters, without any additional monitor or base station elements. This simple architecture can be used to extend the range of the user device in a cost effective manner. For example, a single repeater outfitted with indicators and an alarm snooze button would be an inexpensive way to extend the range and alarm generation capabilities of the user device. Likewise, a network of repeater devices can be dispersed throughout a dwelling to ensure full coverage for alarms, even in the absence of any remote monitoring devices.

The features and components of the systems depicted in FIG. 1 and FIGS. 5-7 can be combined and utilized in different system configurations, and a system embodiment need not be restricted to any of the particular topologies shown in these figures. Indeed, a given system component may have optional user settings that allow the user to customize the data communication capabilities as needed to suit the needs of the particular deployment, dwelling layout, and available device types. That being said, one preferred system embodiment includes the following features and functions: calibration of physiological patient data and the generation of alarms are performed at the ambulatory user device; the user device transmits patient data, alerts, error messages, and the like; any device within the dwelling-based network can receive data transmitted by the user device; any device within the dwelling-based network can re-transmit data to other devices; any device within the dwelling-based network can generate an alarm sound in response to a received alert/alarm message; any device within the dwelling-based network can be used to snooze an alarm; the user device is the only device that is capable of canceling (in contrast to snoozing) certain types of alarms, for example, blood glucose alerts and other critical alarms; and early warning reminders or messages (e.g., “calibration needed in the next two hours” or “change infusion set in eight hours”) can be cancelled at remote devices.

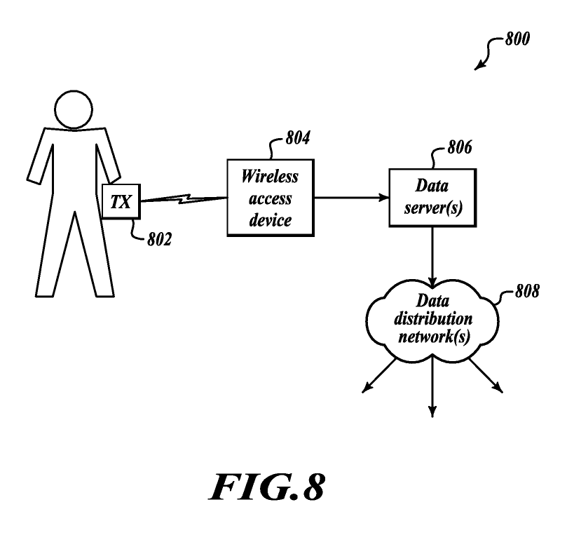

FIG. 8 is a schematic representation of an embodiment of a system 800 for communicating and processing physiological characteristic data for a patient. System 800 generally includes, without limitation: an ambulatory system 802 for processing physiological characteristic data; a wireless access device 804, such as an IEEE 802.11 compliant WLAN access point; one or more data servers 806; and one or more data distribution networks 808.

Ambulatory system 802 may include a self-contained, patient wearable, glucose monitor and wireless telemetry unit, which provides the ability to: continuously monitor the patient's glucose data throughout a healthcare facility or other premises; and continuously communicate the patient's glucose data to data servers 806 or a central monitoring station for continuous remote monitoring of the patient's glucose levels via a wireless network, and/or to a bedside patient monitor from which the glucose data can be displayed on the bedside monitor and communicated to a central monitoring station. Ambulatory system 802 can be embodied in various types of configurations depending upon the desired product feature set. For example, ambulatory system 802 can be configured to support a wireless telemetry mode or a standalone monitoring mode. In the wireless telemetry mode, the ambulatory system 802 functions both as a monitor, continuously monitoring the patient's glucose data in real-time, and as a wireless telemetry device, continuously communicating the patient's glucose data in real-time over a wireless network infrastructure to a data server 806. The data server 806 can then distribute the data to a host of centralized and remote monitoring systems (e.g., a central monitoring station, a wireless hand held PDA or smart phone device, or remote display monitors). Additionally or alternatively, data server 806 can provide the data to hospital clinical information systems, or communicate the data via the internet or an intranet for remote monitoring.

In the standalone monitoring mode ambulatory system 802 continues to monitor the patient's glucose data continuously but without an integrated telemetry radio frequency (RF) module. In this application the ambulatory system 802 provides the ability to continuously monitor the patient's glucose data, and provides both audible and visual indication of the patient's glucose value, high and low alarm limits, and device operational alarms/alerts, but is not capable of wirelessly communicating the data to a wireless network infrastructure on its own.

Certain embodiments of ambulatory system 802 are intended for use in the continuous monitoring and wireless communication physiological patient data of bed ridden patients, ambulatory patients, and intra-hospital transport patients within a hospital patient care ward, extended healthcare facility, and nursing facility. Ambulatory system 802 is capable of being used in the continuous monitoring of patient's glucose data during inter-hospital transport (ground or air), and is capable of communicating the glucose data via a multitude of wireless communication networks including, but not limited to, WANs (e.g., cellular networks) and LANs (e.g., WiFi networks).

Through the use of an integrated display element, user interface controls, memory, and wireless auto-detect circuitry, ambulatory system 802 can continue to monitor the patient's glucose data locally, with alarms in the event that ambulatory system 802 is out of telemetry operating range, or in the event of power loss. In addition, ambulatory system 802 provides clinicians with the ability to remotely monitor physiological characteristics for one or more patients on a continuous real-time basis, and to be immediately alerted to any patient's glucose data that is outside the pre-defined limit ranges (e.g., hyperglycemic or hypoglycemic), and to be alerted to any operational device or sensor status alarms.

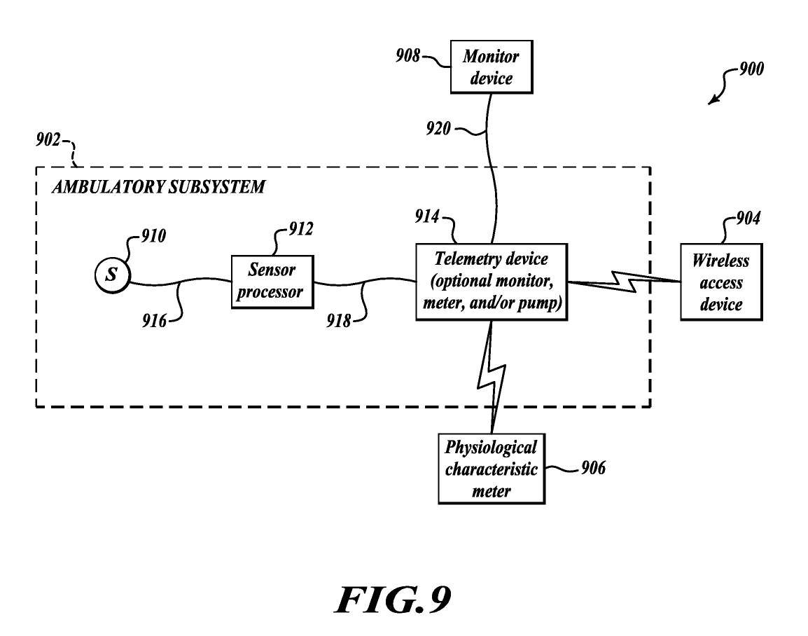

FIG. 9 is a schematic representation of an embodiment of an ambulatory system 900 for communicating and processing physiological characteristic data for a patient. This embodiment of system 900 includes: an ambulatory subsystem 902; a wireless access device 904; an optional physiological characteristic meter 906; and an optional monitor device 908.

Ambulatory subsystem 902 is “ambulatory” in the sense that it is designed to be worn, carried by, or attached to a patient in a manner that allows it to move freely with the patient. In other words, ambulatory subsystem 902 need not be stationary, and it need not rely on any stationary power cables or connections. Ambulatory subsystem 902 includes an ambulatory physiological characteristic sensor 910, a self-contained sensor processor module 912, and an ambulatory telemetry device 914. Sensor 910 is used to measure a physiological characteristic of the patient, such as glucose level. Sensor 910 may be generally configured as described above for physiological characteristic sensor 202 (see FIG. 2). In this embodiment, sensor 910 is configured to generate electrical signals that are indicative of the monitored physiological characteristic of the patient. For example, the electrical signals may have an associated voltage or current that varies in accordance with changes in the monitored physiological characteristic.

Sensor 910 is coupled to sensor processor module 912 in a manner that facilitates transmission of the electrical signals from sensor 910 to sensor processor module 912. The illustrated embodiment employs a sensor lead 916 connected between sensor 910 and sensor processor module 912. Sensor lead 916 includes at least one electrical conductor for the electrical signals generated by sensor 910, and for providing operating power from sensor processor module 912 to sensor 910. Sensor lead 916 can be attached to sensor 910, with a connector for sensor processor module 912 at its free end. Alternatively, sensor lead 916 can be provided as a distinct component with two connectors. In practice, sensor lead 916 is shielded against electromagnetic interference, and the connector end(s) are water tight.

Sensor processor module 912 receives the electrical signals from sensor 910 via sensor lead 916. Sensor processor module 912 is suitably configured to generate measured values of the physiological characteristic from the received electrical signals. As a standalone component, sensor processor module 912 includes its own power supply (battery) that can be recharged by ambulatory telemetry device 914. Sensor processor module 912 also includes its own processing capabilities and memory for data storage. Thus, if the patient needs to travel somewhere without ambulatory telemetry device 914, he can disconnect sensor processor module 912 from ambulatory telemetry device 914, but leave sensor processor module 912 connected to sensor 910. This allows sensor processor module 912 to continue receiving, processing, and storing the sensor data. Eventually, the stored sensor data can be transferred to ambulatory telemetry device 914 at an appropriate time.

The standalone nature of sensor processor module 912 also enables ambulatory telemetry device 914 to be generically configured in the sense that it need not be specifically designed to accommodate any sensor-specific data processing algorithms. Rather, ambulatory telemetry device 914 can be generally designed to relay the already-processed data to other destination devices in the system. Moreover, sensor processor module 912 can be suitably configured for compatibility with other monitoring devices such that the user can transport sensor processor module 912 as needed for connection with remote monitoring devices.

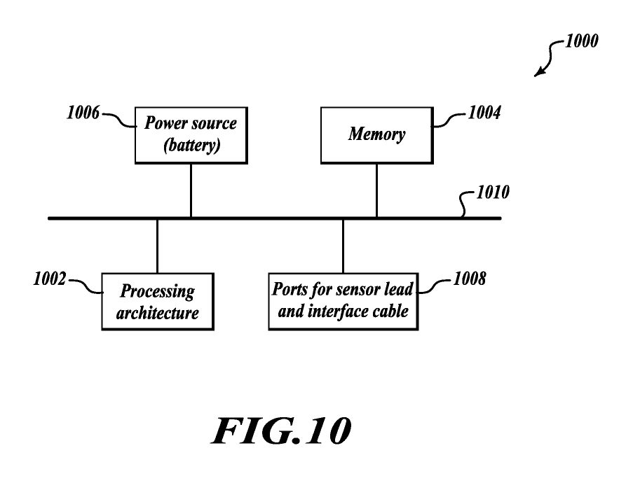

FIG. 10 is a schematic representation of an embodiment of a sensor processor module 1000 suitable for use with system 900. Sensor processor module 1000 is preferably realized as a self-contained component that is physically distinct from the associated sensor and ambulatory telemetry device. Sensor processor module 1000 includes, without limitation: a processing architecture 1002; a suitable amount of memory 1004; a power source 1006; and one or more ports or interfaces 1008 for a sensor lead and/or an interface cable. The elements of sensor processor module 1000 are coupled together by a bus 1010, a conductive architecture, or any suitable interconnection arrangement.

Processing architecture 1002 and memory 1004 may be implemented or performed in the manner described above for processing architecture 316 and memory 314, respectively (see FIG. 3). Processing architecture 1002 is suitably configured as the front end signal processor for electrical signals received from the respective sensor. In practice, processing architecture 1002 performs acquisition, filtering, and conversion of the electrical signals (as detected from the sensor) into glucose values. Memory 1004 can be utilized for the storage of the raw sensor data, processed glucose values, calibration data points (as provided from either an external finger stick glucose meter, an integrated finger stick meter, or manually by a user), glucose trend data, high/low glucose alarm limit settings, or the like.

Power source 1006 can be realized as a removable internal rechargeable battery. Alternatively, power source 1006 might be a hermetically sealed, non-replaceable battery that is rechargeable using inductive charging. Power source 1006 provides operating power for sensor processor module 1000 and, in some embodiments, operating power for the associated ambulatory physiological characteristic sensor. Power source 1006 provides the ability for the sensor processor module 1000 to continue to operate upon disconnection from the host ambulatory telemetry device, and/or upon loss of external power. During the time period when sensor processor module 1000 is not connected to the ambulatory telemetry device, it will continue to provide electrical operating power to the sensor, and it will continue to process and store the patient's glucose data. Upon re-connection with the ambulatory telemetry device, all internally stored data within memory 1004 will be communicated to the ambulatory telemetry device.

Referring again to FIG. 9, ambulatory telemetry device 914 is coupled to self-contained sensor processor module 912 in a manner that facilitates transmission of signals that convey measured values of the physiological characteristic (as generated by sensor processor module 912). The illustrated embodiment employs an interface cable 918 connected between sensor processor module 912 and ambulatory telemetry device 914. Interface cable 918 includes at least one electrical conductor, and interface cable 918 is configured to facilitate data communication between sensor processor module 912 and ambulatory telemetry device 914. Interface cable 918 may also provide operating power from ambulatory telemetry device 914 to sensor processor 912. In one embodiment, interface cable 918 is shielded against electromagnetic interference, and it has a connector for sensor processor module 912 at one end and a connector for ambulatory telemetry device 914 at the other end.

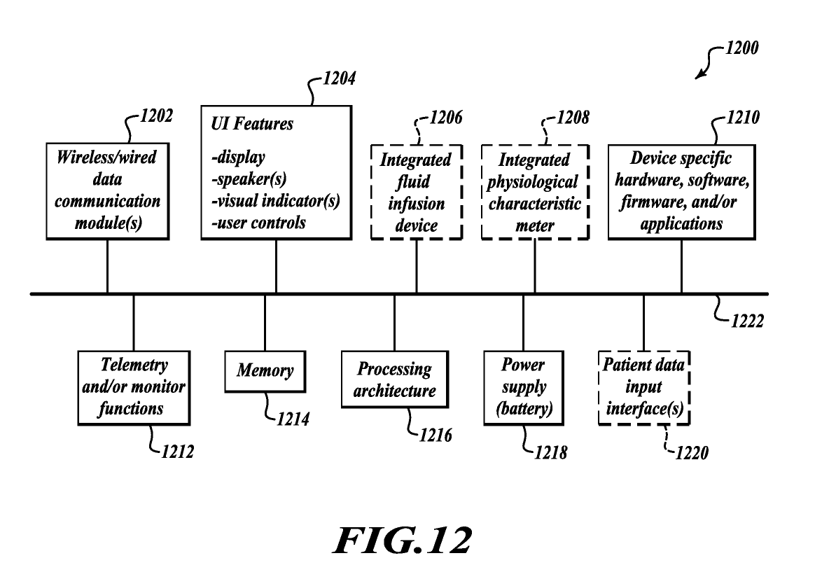

Ambulatory telemetry device 914 receives the measured values of the monitored physiological characteristic (e.g., glucose level), generates sensor signals that convey the measured values, and wirelessly transmits the sensor signals for reception at an appropriate destination device. In system 900, wireless access device 904 represents the destination device. Ambulatory telemetry device 914 represents one embodiment of an ambulatory data receiver device that receives and processes measured values of a physiological characteristic. A generalized ambulatory data receiver device is described in more detail below with reference to FIG. 12.

In one embodiment, ambulatory telemetry device 914 includes hardware, firmware, software, and/or applications that support wireless data telemetry with one or more destination devices. Alternate embodiments may include one or more optional features. For example, ambulatory telemetry device 914 may be suitably configured to also function as an ambulatory monitor device for the patient. In such an embodiment, ambulatory telemetry device 914 includes an integrated monitor that produces audio/visual indicia associated with the measured values, device or system status information, or the like. In practice, this integrated ambulatory monitor can include any of the monitor features described in more detail herein. Ambulatory telemetry device 914 may also include an (optional) ambulatory fluid infusion device for the patient, such as an insulin pump. For such an embodiment, ambulatory telemetry device 914 can process and transmit status data, alarms, and/or other information related to the operation of the integrated fluid infusion device. Ambulatory telemetry device 914 may also include an (optional) integrated meter device that is configured to directly measure the physiological characteristic of the patient. The integrated meter device enables ambulatory telemetry device 914 to process the direct measurements along with the measured values obtained from sensor 910, for purposes of calibration, redundancy, etc. In one particular embodiment, the integrated meter device is realized as a BG meter that receives and analyzes blood sample test strips. Such “fingerstick” BG meters are well known and, therefore, will not be described in detail here.

As mentioned above, ambulatory telemetry device 914 may be configured to cooperate with an external monitor device 908 (this is another optional feature). The illustrated embodiment shows monitor device 908 connected to ambulatory telemetry device 914 through an interface cable 920. Alternatively or additionally, monitor device 908 can be coupled to ambulatory telemetry device 914 via one or more wireless links. In practice, monitor device 908 receives signals (e.g., sensor signals that convey the measured values of the monitored physiological characteristic) from ambulatory telemetry device 914 via interface cable 920, processes the received signals, and generates audio/visual indicia in response to the received signals. In practice, monitor device 908 can include any of the monitor features described in more detail herein, and monitor device 908 can generate indicia of items such as the measured values, alarms, device or system status, reminders, or the like.

System 900 may include an optional physiological characteristic meter device 906, which is suitably configured to directly measure a physiological characteristic of interest (e.g., glucose level), and to transmit such directly-measured values of the physiological characteristic to ambulatory telemetry device 914. The illustrated embodiment depicts meter device 906 as a wireless device that contains a wireless transmitter/transceiver that supports wireless transmission of data with ambulatory telemetry device 914. Alternatively or additionally, meter device 906 can be coupled to ambulatory telemetry device 914 via a physical connection, such as an interface cable. Ambulatory telemetry device 914 can process the direct measurements received from meter device 906, along with the measured values obtained from sensor 910, for purposes of calibration, redundancy, etc. In one particular embodiment, meter device 906 is realized as a BG meter that receives and analyzes blood sample test strips. Such “fingerstick” BG meters are well known and, therefore, will not be described in detail here.

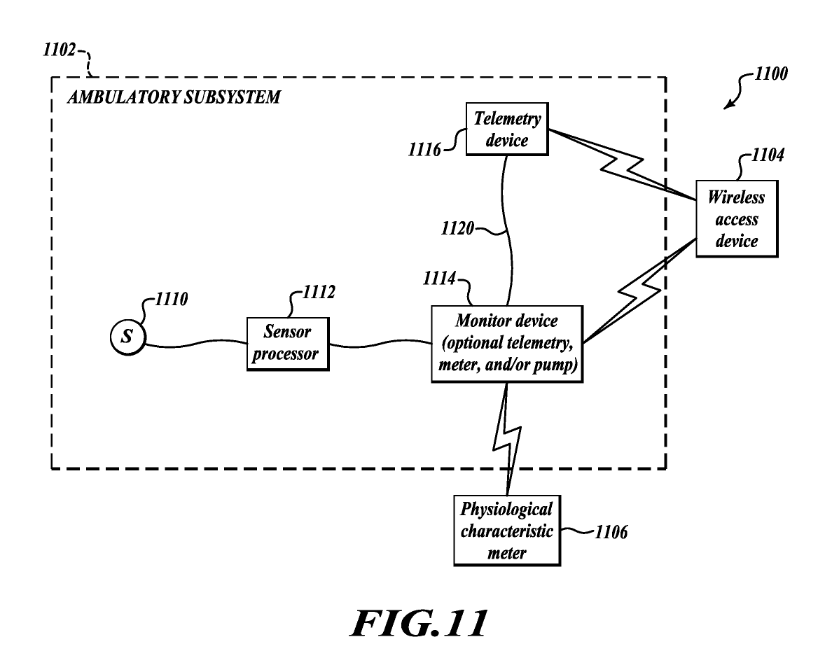

Ambulatory subsystem 902 employs a host device that primarily functions as a telemetry device. Alternate system embodiments can instead utilize a host device that primarily functions as a monitor device. In this regard, FIG. 11 is a schematic representation of an alternate embodiment of an ambulatory system 1100 for communicating and processing physiological characteristic data for a patient. This embodiment of system 1100 includes: an ambulatory subsystem 1102; an optional wireless access device 1104; and an optional physiological characteristic meter 1106. This embodiment of ambulatory subsystem 1102 includes, without limitation: an ambulatory physiological characteristic sensor 1110; a self-contained sensor processor module 1112; an ambulatory monitor device 1114; and an optional ambulatory telemetry device 1116. Some of the components in system 1100 are identical, similar, or equivalent to counterpart components in system 900. For the sake of brevity, common features, functions, and characteristics will not be redundantly described here in the context of system 1100.

Ambulatory monitor device 1114 is coupled to sensor processor module 1112 such that it can receive the measured values of the monitored physiological characteristic (e.g., glucose level) from sensor processor module 1112, process the measured values, and produce audio/visual indicia associated with the measured values. Ambulatory monitor device 1114 represents one embodiment of an ambulatory data receiver device that receives and processes measured values of a physiological characteristic. A generalized ambulatory data receiver device is described in more detail below with reference to FIG. 12.

In one embodiment, ambulatory monitor device 1114 includes hardware, firmware, software, and/or applications that support patient monitoring features and functions. In practice, ambulatory monitor device 1114 can include any of the monitor features, functions, and options described in more detail herein, and ambulatory monitor device 1114 can generate indicia of items such as the measured values, alarms, device or system status, reminders, or the like. For instance, ambulatory monitor device 1114 can continuously monitor and display the patient glucose values (as detected by sensor 1110, processed by sensor processor module 1112, and communicated to ambulatory monitor device 1114). Ambulatory monitor device 1114 can also display retrospective trend data, glucose alarm limits (high and/or low), calibration reference values (as measured by either an external finger stick meter, internal finger stick meter, or manually entered), and operational status alarms for the devices in the system.

Alternate embodiments of ambulatory monitor device 1114 may include one or more optional features. For example, ambulatory monitor device 1114 may be suitably configured to also function as an ambulatory fluid infusion device for the patient and/or to also function as an integrated meter device that directly measures the physiological characteristic of interest (these optional features were described above in the context of ambulatory telemetry device 914). Ambulatory monitor device 1114 may also include an (optional) ambulatory telemetry device integrated therein, where the integrated telemetry function is suitably configured to generate sensor signals that convey the measured values of the physiological characteristic of interest, and to wirelessly transmit the sensor signals for reception at a compatible destination device. Accordingly, the primary telemetry functionality of ambulatory telemetry device 914 can be incorporated into ambulatory monitor device 1114 to implement this option. For the embodiment of system 1100 illustrated in FIG. 11, the optional wireless access device 1104 represents a destination device that wirelessly communicates with ambulatory monitor device 1114.

As mentioned above, ambulatory monitor device 1114 may be configured to cooperate with an external ambulatory telemetry device 1116 (this is another optional feature). This ambulatory telemetry device 1116 is “external” relative to ambulatory monitor device 1114 because it is a physically distinct component. However, ambulatory telemetry device 1116 is “ambulatory” in the sense that it is designed to be worn, carried, or attached to the patient in a manner that allows it to move about from place to place along with the patient. Ambulatory telemetry device 1116 may be configured to support any of the features, functions, and operations supported by ambulatory telemetry device 914. Moreover, ambulatory telemetry device 1116 may be realized as a single-parameter device (e.g., a device that only handles glucose data obtained from ambulatory monitor device 1114), or as a multi-parameter device that has one or more additional patient data input interfaces for receiving additional physiological patient data corresponding to different physiological characteristics (e.g., data other than glucose levels).

The illustrated embodiment shows ambulatory telemetry device 1116 connected to ambulatory monitor device 1114 through an interface cable 1120. Alternatively or additionally, ambulatory telemetry device 1116 can be coupled to ambulatory monitor device 1114 via one or more wireless links. In this example, ambulatory telemetry device 1116 receives data from ambulatory monitor device 1114 via interface cable 1120, processes the received data, and transmits wireless signals to a compatible destination device, such as wireless access device 1104. Thereafter, the data received at the destination device can be further processed, routed, or communicated as desired.

System 1100 may include an optional physiological characteristic meter device 1106, which is suitably configured to directly measure a physiological characteristic of interest (e.g., glucose level), and to transmit such directly-measured values of the physiological characteristic to ambulatory monitor device 1114. The above description of meter device 906 also applies to meter device 1106.

As mentioned above, ambulatory telemetry device 914 (see FIG. 9) and ambulatory monitor device 1114 (see FIG. 11) represent two different embodiments of an ambulatory data receiver device that receives and processes measured values of the physiological characteristic. FIG. 12 is a schematic representation of a generalized ambulatory data receiver device 1200 suitable for use with an ambulatory system for communicating and processing physiological characteristic data for a patient. This embodiment of ambulatory data receiver device 1200 includes, without limitation: wireless/wired data communication module(s) 1202; one or more UI features 1204; an optional integrated fluid infusion device 1206; an optional integrated physiological characteristic meter 1208; device specific hardware, software, firmware, and/or applications 1210; telemetry and/or monitor functions 1212; a suitable amount of memory 1214; a processing architecture 1216; a power supply 1218; and one or more optional patient data input interfaces 1220. The elements of ambulatory data receiver device 1200 may be coupled together via a bus 1222 or any suitable interconnection architecture.

Processing architecture 1216 and memory 1214 may be implemented or performed in the manner described above for processing architecture 316 and memory 314, respectively (see FIG. 3). Device-specific hardware, software, firmware, and/or applications 1210 may vary from one embodiment of ambulatory data receiver device 1200 to another. Again, one embodiment supports at least the primary telemetry functions, while another embodiment supports at least the primary monitor functions (represented by telemetry and/or monitor functions 1212). In practice, an embodiment may combine one or more of: telemetry functions; monitor functions; integrated fluid infusion device 1206; integrated physiological characteristic meter 1208; and possibly other features. Accordingly, device-specific hardware, software, firmware, and/or applications 1210 will support the particular set of features implemented by ambulatory data receiver device 1200. In practice, certain portions or aspects of device-specific hardware, software, firmware, and/or applications 1210 may be implemented in one or more of the other blocks depicted in FIG. 12.

An embodiment of ambulatory data receiver device 1200 may employ any number of wireless and/or wired data communication modules 1202. These data communication modules are suitably configured to support wireless/wired data communication (unidirectional or bidirectional, depending upon the particular implementation) between ambulatory data receiver device 1200 and other devices in the system, for example, a sensor processor module, an external physiological characteristic meter, an external monitor device, an external telemetry device, a wireless access device, a computing device, etc. Wireless/wired data communication modules 1202 may be configured as generally described above in the context of wireless/wired data communication modules 302 (see FIG. 3). In practice, wireless/wired data communication modules 1202 may include (or be realized as) a data input/output port that is used to connect ambulatory data receiver device 1200 to an external data gathering, data processing, data recording, or data communication device, network, or architecture. In this manner, ambulatory data receiver device 1200 can support wireless and/or wired data communication with remote network devices using appropriate data communication protocols.

Ambulatory data receiver device 1200 may also be designed to accommodate various UI features 1204 that allow the user to control and interact with ambulatory data receiver device 1200 and/or other devices within the system. In practice, UI features 1204 may include any number of the UI elements, components, or features described above, including, without limitation: a display element; speakers; visual indicators; and user controls.

Patient data input interfaces 1220 are configured for receiving additional data signals that are indicative of one or more additional physiological characteristics of the patient. For example, patient data input interfaces 1220 may be designed for compatibility with existing connector types or standard connector configurations that are employed in the medical device and equipment industry. In particular, patient data input interfaces 1220 may be suitably configured to accommodate: ECG lead wires that can be affixed to the patient; pulse oximetry sensors; respiration sensors; thermometers; blood pressure equipment; or the like. Thus, patient data input interfaces 1220 enable an embodiment of ambulatory data receiver device 1200 to operate in a multi-parameter mode.

Power supply 1218 may include one or more batteries, including a main system removable rechargeable battery, which is used to provide operational power for ambulatory data receiver device 1200 and the sensor processor module, and a smaller backup battery, which is used to provide temporary operating power for ambulatory data receiver device 1200 battery exchange periods. The internal backup battery is automatically selected when the main battery is either very low or is removed from ambulatory data receiver device 1200. Upon insertion of a fully charged battery, the backup battery is switched out and begins to recharge from the main battery. Some embodiments of ambulatory data receiver device 1200 include a battery charging docking unit that can be used to recharge the backup battery and/or the main battery.

In summary, systems, devices, and methods configured in accordance with the embodiments described herein relate to:

(1) A system for remote wireless monitoring of data for a patient, where the system comprises: an ambulatory sensor/transmitter subsystem configured to obtain measured values of a physiological characteristic of the patient, and to wirelessly transmit sensor signals that convey the measured values; a base station in wireless communication with the ambulatory sensor/transmitter subsystem, the base station being configured to wirelessly receive the sensor signals, generate base station signals in response to the sensor signals, and wirelessly transmit the base station signals; and a remote monitor in wireless communication with the base station, the remote monitor being configured to wirelessly receive the base station signals, and generate audio/visual indicia in response to the base station signals.

The system may further comprise a repeater in wireless communication with the base station and in wireless communication with the remote monitor, the repeater being configured to wirelessly receive the base station signals from the base station, and wirelessly retransmit the base station signals to the remote monitor.

In this system, the remote monitor may comprise a repeater, the repeater being configured to wirelessly receive the base station signals from the base station, and wirelessly retransmit the base station signals to a receiving device.

In this system, the ambulatory sensor/transmitter subsystem may comprise: a physiological characteristic sensor; a sensor transmitter coupled to the physiological characteristic sensor, the sensor transmitter being configured to wirelessly transmit sensor origination signals; and a telemetry device in wireless communication with the sensor transmitter, the telemetry device being configured to wirelessly receive the sensor origination signals, generate the sensor signals from the sensor origination signals, and wirelessly transmit the sensor signals. The telemetry device may comprise an ambulatory monitor device for the patient, the ambulatory monitor device being configured to produce audio/visual indicia associated with the measured values. The telemetry device may comprise an ambulatory fluid infusion device for the patient.

In this system, the base station may comprise a communication module configured to facilitate data communication using an external communication network. The communication module may be configured to facilitate data communication using a cellular telecommunication network, a wide area network, or a local area network.

In this system, the ambulatory sensor/transmitter subsystem can be configured to wirelessly transmit sensor signals using a relatively short range wireless data communication protocol; and the base station can be configured to wirelessly transmit base station signals using a relatively long range wireless data communication protocol.

In this system, the remote monitor may comprise a mobile computing device or a mobile communication device.

In this system, the audio/visual indicia may comprise an audio/visual representation of the measured values, an audio/visual representation of an alarm triggered by the measured values, an audio/visual representation of an operating status of the ambulatory sensor/transmitter subsystem, or an audio/visual representation of an operating status of the base station.

(2) A system for remote wireless monitoring of data for a patient, where the system comprises: an ambulatory telemetry device configured to wirelessly transmit physiological characteristic data for the patient; a base station in wireless communication with the ambulatory telemetry device, the base station being configured to wirelessly receive the physiological characteristic data, generate base station signals in response to the physiological characteristic data, and wirelessly transmit the base station signals; and a remote monitor in wireless communication with the base station, the remote monitor being configured to wirelessly receive the base station signals, and generate audio/visual indicia in response to the base station signals.

The ambulatory telemetry device may comprise an ambulatory monitor device for the patient, the ambulatory monitor device being configured to produce audio/visual indicia associated with the physiological characteristic data. In another embodiment, the ambulatory telemetry device comprises an ambulatory fluid infusion device for the patient.

The system may further comprise an ambulatory sensor/transmitter subsystem in wireless communication with the ambulatory telemetry device, the ambulatory sensor/transmitter subsystem being configured to measure a physiological characteristic of the patient and to wirelessly transmit sensor signals that convey measured values of the physiological characteristic, wherein the ambulatory telemetry device is configured to wirelessly receive the sensor signals, and generate the physiological characteristic data in response to the sensor signals.

(3) A system for remote wireless monitoring of data for a patient, where the system comprises: a transmitting device configured to wirelessly transmit patient data signals that convey measured values of a physiological characteristic of the patient; and a plurality of wireless remote units for the transmitting device, the plurality of wireless remote units being cooperatively configured to operate as a wireless repeater network for the patient data signals; wherein the plurality of wireless remote units comprises a wireless remote monitor in communication with the transmitting device, the wireless remote monitor being configured to wirelessly receive the patient data signals or retransmitted versions thereof, and generate audio/visual indicia of the measured values.

The transmitting device may comprise: an ambulatory sensor/transmitter subsystem that is configured to measure the physiological characteristic of the patient; an ambulatory monitor device for the patient, the ambulatory monitor device being configured to produce audio/visual indicia of the measured values; an ambulatory fluid infusion device for the patient; or an ambulatory monitor/pump device for the patient.

(4) A method for remote wireless monitoring of data for a patient, the method comprising: measuring a physiological characteristic of the patient; wirelessly transmitting a sensor signal that conveys a measured value of the physiological characteristic; wirelessly receiving the sensor signal at a base station; generating a base station signal in response to the sensor signal; wirelessly transmitting the base station signal from the base station; and wirelessly receiving the base station signal, or a retransmitted version thereof, at a remote monitor.

The method may further comprise: wirelessly receiving the base station signal at a repeater; and wirelessly retransmitting the base station signal from the repeater, wherein the remote monitor wirelessly receives a retransmitted version of the base station signal from the repeater.

The method may further comprise the remote monitor generating, in response to the base station signal received at the remote monitor, audio/visual indicia of the measured value.

The method may further comprise the base station transmitting, in response to the sensor signal received at the base station, an external network data communication signal that conveys the measured value.

In certain embodiments of this method, wirelessly transmitting a sensor signal that conveys a measured value of the physiological characteristic is performed in compliance with a relatively short range wireless data communication protocol; wirelessly receiving the sensor signal at a base station is performed in compliance with the relatively short range wireless data communication protocol; wirelessly transmitting the base station signal from the base station is performed in compliance with a relatively long range wireless data communication protocol; and wirelessly receiving the base station signal, or a retransmitted version thereof, at a remote monitor is performed in compliance with the relatively long range wireless data communication protocol.

(5) A wireless repeater for a system that remotely monitors patient data, the wireless repeater comprising: a receiver configured to wirelessly receive sensor signals that convey measured values of a physiological characteristic of a patient; a transmitter coupled to the receiver, and configured to wirelessly retransmit the sensor signals; a signal analyzer coupled to the receiver, and configured to analyze characteristics and content of received signals; a wireless signal strength indicator coupled to the signal analyzer, and configured to generate audio/visual indicia of received signal strength while the wireless repeater is operating in a setup mode; and a data throughput indicator coupled to the signal analyzer, and configured to generate audio/visual indicia of wirelessly received data while the wireless repeater is operating in the setup mode.

The data throughput indicator may be configured to generate audio/visual indicia of received sensor signals.

The wireless repeater may further comprise an alarm indicator coupled to the signal analyzer, the alarm indicator being configured to generate an audio/visual alarm in response to received sensor signals. This wireless repeater may further comprise an alarm snooze control that is configured to disable the audio/visual alarm.

(6) An ambulatory system for processing physiological characteristic data for a patient, the system comprising: an ambulatory physiological characteristic sensor configured to generate electrical signals that are indicative of a physiological characteristic of the patient; a self-contained sensor processor module coupled to the ambulatory physiological characteristic sensor, the self-contained sensor processor module being configured to receive the electrical signals from the ambulatory physiological characteristic sensor, and generate measured values of the physiological characteristic from the electrical signals; and an ambulatory telemetry device coupled to the self-contained sensor processor module, the ambulatory telemetry device being configured to receive the measured values, generate sensor signals that convey the measured values, and wirelessly transmit the sensor signals for reception at a destination device.

In this system, the ambulatory telemetry device may comprise: an ambulatory monitor device for the patient, the ambulatory monitor device being configured to produce audio/visual indicia associated with the measured values; or an ambulatory fluid infusion device for the patient.

This system may further comprise a sensor lead connected between the ambulatory physiological characteristic sensor and the self-contained sensor processor module, the sensor lead comprising an electrical conductor for the electrical signals.

In this system, the self-contained sensor processor module may further comprise a power source that provides operating power for the ambulatory physiological characteristic sensor.

The system may further comprise an interface cable connected between the self-contained sensor processor module and the ambulatory telemetry device, the interface cable being configured to facilitate data communication between the self-contained sensor processor module and the ambulatory telemetry device.

The system may further comprise a meter device configured to directly measure the physiological characteristic of the patient, and transmit directly-measured values of the physiological characteristic to the ambulatory telemetry device. For this system, the meter device may comprise a wireless transmitter configured to wirelessly transmit the directly-measured values to the ambulatory telemetry device.

The ambulatory telemetry device may comprise an integrated meter device that is configured to directly measure the physiological characteristic of the patient.

The system may further comprise: a monitor device; and an interface cable that connects the monitor device to the ambulatory telemetry device, the monitor device being configured to receive the sensor signals from the ambulatory telemetry device via the interface cable, and to generate audio/visual indicia in response to the sensor signals.

The ambulatory telemetry device may comprise a patient data input interface for receiving a second signal that is indicative of a second physiological characteristic of the patient.

(7) An ambulatory system for processing physiological characteristic data for a patient, the system comprising: an ambulatory physiological characteristic sensor configured to generate electrical signals that are indicative of a physiological characteristic of the patient; a self-contained sensor processor module coupled to the ambulatory physiological characteristic sensor, the self-contained sensor processor module being configured to receive the electrical signals from the ambulatory physiological characteristic sensor, and generate measured values of the physiological characteristic from the electrical signals; and an ambulatory monitor device coupled to the self-contained sensor processor module, the ambulatory monitor device being configured to receive the measured values, and produce audio/visual indicia associated with the measured values.

In this system, the ambulatory monitor device may comprise an ambulatory telemetry device that is configured to generate sensor signals that convey the measured values, and wirelessly transmit the sensor signals for reception at a destination device.

The system may further comprise an ambulatory telemetry device coupled to the ambulatory monitor device.

The ambulatory monitor device may comprise an ambulatory fluid infusion device for the patient.

The system may further comprise an interface cable connected between the self-contained sensor processor module and the ambulatory monitor device, the interface cable being configured to facilitate data communication between the self-contained sensor processor module and the ambulatory monitor device.

The system may further comprise a meter device configured to directly measure the physiological characteristic of the patient, and transmit directly-measured values of the physiological characteristic to the ambulatory monitor device. In this system, the meter device may comprise a wireless transmitter configured to wirelessly transmit the directly-measured values to the ambulatory monitor device.

The ambulatory monitor device may comprise an integrated meter device that is configured to directly measure the physiological characteristic of the patient.

The ambulatory monitor device may comprise a patient data input interface for receiving a second signal that is indicative of a second physiological characteristic of the patient.

(8) In a system comprising an ambulatory physiological characteristic sensor, an ambulatory data receiver device, and a self-contained sensor processor module coupled between the ambulatory physiological characteristic sensor and the ambulatory data receiver device, a method for communicating physiological characteristic data for a patient. The method involves: generating, with the ambulatory physiological characteristic sensor, electrical signals that are indicative of a physiological characteristic of the patient; receiving the electrical signals at the self-contained sensor processor module; generating, with the self-contained sensor processor module, measured values of the physiological characteristic from the electrical signals; and receiving the measured values at the ambulatory data receiver.

The method may further comprise producing audio/visual indicia of the measured values at the ambulatory data receiver.

The method may further comprise: generating, with the ambulatory data receiver, sensor signals that convey the measured values; and wirelessly transmitting the sensor signals for reception at a destination device.

In this method, receiving the electrical signals may comprise wirelessly receiving the electrical signals from the ambulatory physiological characteristic sensor.

In this method, receiving the measured values may comprise wirelessly receiving the measured values from the self-contained sensor processor module.

The method may further comprise receiving directly-measured values of the physiological characteristic at the ambulatory data receiver, the directly-measured values being directly measured by a meter device that is distinct from the ambulatory data receiver.

The method may further comprise obtaining, with the ambulatory data receiver, directly-measured values of the physiological characteristic.

The method may further comprise: generating, with the ambulatory data receiver, sensor signals that convey the measured values; and transmitting the sensor signals over an external communication network to a destination device.

While at least one example embodiment has been presented in the foregoing detailed description, it should be appreciated that a vast number of variations exist. It should also be appreciated that the example embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A system for remote wireless monitoring of data for a patient, the system comprising:
an ambulatory sensor/transmitter subsystem configured to obtain measured values of a physiological characteristic of the patient, and to wirelessly transmit sensor signals that convey the measured values;
a base station in wireless communication with the ambulatory sensor/transmitter subsystem, the base station being configured to wirelessly receive the sensor signals, generate base station signals in response to the sensor signals, and wirelessly transmit the base station signals; and
a remote monitor in wireless communication with the base station, the remote monitor being configured to wirelessly receive the base station signals, and generate audio/visual indicia in response to the base station signals.

2. The system of claim 1, further comprising a repeater in wireless communication with the base station and in wireless communication with the remote monitor, the repeater being configured to wirelessly receive the base station signals from the base station, and wirelessly retransmit the base station signals to the remote monitor.

3. The system of claim 1, the remote monitor comprising a repeater, the repeater being configured to wirelessly receive the base station signals from the base station, and wirelessly retransmit the base station signals to a receiving device.

4. The system of claim 1, the ambulatory sensor/transmitter subsystem comprising:
a physiological characteristic sensor;
a sensor transmitter coupled to the physiological characteristic sensor, the sensor transmitter being configured to wirelessly transmit sensor origination signals; and
a telemetry device in wireless communication with the sensor transmitter, the telemetry device being configured to wirelessly receive the sensor origination signals, generate the sensor signals from the sensor origination signals, and wirelessly transmit the sensor signals.

5. The system of claim 4, the telemetry device comprising an ambulatory monitor device for the patient, the ambulatory monitor device being configured to produce audio/visual indicia associated with the measured values.

6. The system of claim 4, the telemetry device comprising an ambulatory fluid infusion device for the patient.

7. The system of claim 1, the base station comprising a communication module configured to facilitate data communication using a network selected from the group consisting of an external communication network, a cellular telecommunication network, a wide area network, and a local area network.

8. The system of claim 1, wherein:
the ambulatory sensor/transmitter subsystem is configured to wirelessly transmit sensor signals using a relatively short range wireless data communication protocol; and
the base station is configured to wirelessly transmit base station signals using a relatively long range wireless data communication protocol.

9. The system of claim 1, the remote monitor comprising a mobile computing device.

10. A wireless repeater for a system that remotely monitors patient data, the wireless repeater comprising:
a receiver configured to wirelessly receive sensor signals that convey measured values of a physiological characteristic of a patient;
a transmitter coupled to the receiver, and configured to wirelessly retransmit the sensor signals;
a signal analyzer coupled to the receiver, and configured to analyze characteristics and content of received signals;
a wireless signal strength indicator coupled to the signal analyzer, and configured to generate audio/visual indicia of received signal strength while the wireless repeater is operating in a setup mode; and
a data throughput indicator coupled to the signal analyzer, and configured to generate audio/visual indicia of wirelessly received data.

11. The wireless repeater of claim 10, the data throughput indicator being configured to generate audio/visual indicia of received sensor signals.

12. The wireless repeater of claim 10, further comprising an alarm indicator coupled to the signal analyzer, the alarm indicator being configured to generate an audio/visual alarm in response to received sensor signals.

13. The wireless repeater of claim 12, further comprising an alarm snooze control that is configured to disable the audio/visual alarm.

14. An ambulatory system for processing physiological characteristic data for a patient, the system comprising:
an ambulatory physiological characteristic sensor configured to generate electrical signals that are indicative of a physiological characteristic of the patient;
a self-contained sensor processor module coupled to the ambulatory physiological characteristic sensor, the self-contained sensor processor module being configured to receive the electrical signals from the ambulatory physiological characteristic sensor, and generate measured values of the physiological characteristic from the electrical signals; and
an ambulatory telemetry device coupled to the self-contained sensor processor module, the ambulatory telemetry device being configured to receive the measured values, generate sensor signals that convey the measured values, and wirelessly transmit the sensor signals for reception at a destination device.

15. The system of claim 14, the ambulatory telemetry device comprising an ambulatory monitor device for the patient, the ambulatory monitor device being configured to produce audio/visual indicia associated with the measured values.

16. The system of claim 14, the ambulatory telemetry device comprising an ambulatory fluid infusion device for the patient.

17. The system of claim 14, further comprising an interface cable connected between the self-contained sensor processor module and the ambulatory telemetry device, the interface cable being configured to facilitate data communication between the self-contained sensor processor module and the ambulatory telemetry device.

18. The system of claim 14, further comprising a meter device configured to directly measure the physiological characteristic of the patient, and transmit directly-measured values of the physiological characteristic to the ambulatory telemetry device.

19. The system of claim 18, the meter device comprising a wireless transmitter configured to wirelessly transmit the directly-measured values to the ambulatory telemetry device.

20. The system of claim 14, the ambulatory telemetry device comprising an integrated meter device that is configured to directly measure the physiological characteristic of the patient.
